(19) United States
(12) Patent Application Publication
(10) Pub. No.: US 2017/0137868 A1
(43) Pub. Date: May 18, 2017

(54) PROCESS CONTROL STRAINS AND METHODS OF DETECTING

(71) Applicant: REGENTS OF THE UNIVERSITY OF MINNESOTA, MINNEAPOLIS, MN (US)

(72) Inventors: Satoshi Ishii, Roseville, MN (US); Qian Zhang, St. Paul, MN (US)

(21) Appl. No.: 15/347,845

(22) Filed: Nov. 10, 2016

Related U.S. Application Data
(60) Provisional application No. 62/254,924, filed on Nov. 13, 2015.

(51) Int. Cl. C12Q 1/68

(52) U.S. Cl. CPC C12Q 1/689

(57) ABSTRACT

Provided herein are methods for determining the concentration of a microbe in a sample using a genetically engineered microbe as a process control strain. The sample is one that is suspected of including a test microbe, such as a microbe that is a contaminant of an environmental or clinical sample. A known amount of the process control strain is added to the sample and DNA of microbes present is extracted and amplified. The DNA recovery efficiency of the genetically engineered microbe is determined and used to determine the number of cells of the test microbe in the sample. Also provided are kits and genetically engineered microbes useful as process control strains.

### CROSS-REFERENCE TO RELATED APPLICATIONS

This application claims the benefit of U.S. Provisional Application Ser. No. 62/254,924, filed Nov. 13, 2015, which is incorporated by reference herein.

### SEQUENCE LISTING

This application contains a Sequence Listing electronically submitted via EFS-Web to the United States Patent and Trademark Office as an ASCII text file entitled “11005250101_SequenceListing_ST25.txt” having a size of 16 kilobytes and created on Nov. 7, 2016. The information contained in the Sequence Listing is incorporated by reference herein.

### SUMMARY OF THE APPLICATION

Provided herein are methods, including methods for determining the concentration of a microbe in a sample. In one embodiment the method includes extracting DNA of microbes present in a sample, where the sample includes a known number of cells of a genetically engineered microbe, and where the sample is suspected of including a test microbe. Primers are added to the sample, where the primers include (1) primers to amplify DNA of the genetically engineered microbe and (2) primers to amplify DNA of the test microbe. The sample is exposed to conditions suitable for amplification of a target polynucleotide. The target polynucleotide includes a target polynucleotide of the genetically engineered microbe, a target polynucleotide of the test microbe, or a combination thereof. The DNA recovery efficiency of the genetically engineered microbe is determined, and the number of cells of the test microbe in the sample is calculated. In one embodiment, it is determined that the sample does not include a test microbe.

In one embodiment, the method can further include adding to the sample a predetermined number of cells of the genetically engineered microbe. In one embodiment, the method further includes concentrating the microbes present in the sample. In one embodiment, the genetically engineered microbe and the test microbe are Gram negative microbes. In another embodiment, the genetically engineered microbe and the test microbe are Gram positive microbes.

In one embodiment, the sample is divided into at least two aliquots, and the primers that amplify DNA of the genetically engineered microbe are added to one aliquot and the primers that amplify DNA of the test microbe are added to a separate aliquot.

The sample can include an environmental sample, such as recreational water. The sample can include a clinical sample, such as tissue, stool, or a body fluid. The sample can include a food sample, such as meat, fish, mild, cheese, fruit, or vegetable. In another embodiment, the sample includes groundwater, leachate, wastewater, sewer water, blackwater, graywater, bilge water, ballast water, feed water, process water, industrial water, irrigation water, rain water, runoff water, cooling water, non-potable water, potable water, or drinking water.

In another embodiment the method includes extracting DNA of microbes present in a sample, wherein the sample includes a known number of cells of a genetically engineered microbe, and wherein the sample is suspected of including a test microbe. Primers are added to the sample, wherein the primers include (1) primers to amplify DNA of the genetically engineered microbe, and (2) primers to amplify DNA of the test microbe. A target polynucleotide of the genetically engineered microbe is amplified and a target polynucleotide of the test microbe is amplified. The DNA recovery efficiency of the genetically engineered microbe is determined, and the number of cells of the test microbe in the sample is calculated.

Also provided are kits. In one embodiment, a kit includes in separate containers a genetically engineered microbe and primers. The microbe is not a member of the microbiota of a human or an animal, and the primers can be used to amplify a target polynucleotide present in the genetically engineered microbe. In one embodiment, the test microbe is Campylobacter jejuni, Campylobacter lari, Listeria monocytogenes, Salmonella spp., Shigella spp., Clostridium perfringens, Legionella pneumophila, Listeria monocytogenes, Vibrio cholera, Vibrio paraheamolyticus, or E. coli. Examples of E. coli include, but are not limited to, E. coli O157:H7, a Shiga-toxin producing E. coli, and an enteropathogenic E. coli. In one embodiment, the test microbe is a microbe indicative of fecal contamination of a water sample, such as Enterococcus spp. or Bacteroides spp.

Further provided is a genetically engineered microbe. In one embodiment, the genetically engineered microbe is Pseudogulbenkiania spp., Pantoea stewartii subsp. stewartii, or a member of the genus Geobacillus, such as G. thermodenitrificans. The genetically engineered microbe includes an exogenous polynucleotide integrated in the genome. In one embodiment, the exogenous polynucleotide is at least 20 nucleotides in length. In one embodiment, the exogenous polynucleotide is integrated in nucleotides encoding a ribosomal component of the large or small subunit of a prokaryotic ribosome, such as 16S or 23S, of the genetically engineered microbe. In one embodiment, the exogenous polynucleotide includes a transposon.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows the location of the Tn5RL27 insertion in Pseudogulbenkiania sp. strain NH8B-1D2. Transposon Tn5RL27 was inserted into one of the eight 23S rRNA genes (NH8B_3960) of Pseudogulbenkiania sp. strain NH8B-1D2. Numbers show the location (in base pair) on the genome (GenBank accession AP012224).

FIG. 2 shows the whole sequence of pRL27-NH8B-1D2-NcoI-1. Sequences of Tn5RL27 is shown in capital letters.

FIG. 3 shows annealing sites of the qPCR assays for specific quantification of strain NH8B-1D2. Two assays (NH8B_3960tnp1 and NH8B_3960tnp2) were designed in the junction regions between NH8B_3960 and Tn5RL27 genes.

FIG. 4 shows a standard curve generated by conventional qPCR assay (NH8B_3960tnp1).

FIG. 5 shows a standard curve generated by MFQPCR assay (NH8B_3960tnp1).

FIG. 6 shows a heat-map generated based on the C_{T} value obtained by MFQPCR. Twenty four genes targeting various bacteria, including Pseudogulbenkiania sp. strain NH8B-1D2, were simultaneously quantified.

FIG. 7 shows relationship between concentrations of pathogens measured by MFQPCR and normalized by using SPC (Q^{N}_{PATH}) and the actual concentrations of pathogens (I_{PATH}; the concentration of pathogens spiked to the environmental samples). Four pathogens (Escherichia coli O157:H7 strain Sakai (MMD 0509952), Salmonella enterica serovar Typhimurium JCM1652^{T}, Campylobacter jejuni JCM2013^{T}, and Listeria monocytogenes serovar 1/2a JCM 7671) were tested in this study. Linear regression for each pathogen is shown in Table 7.

### DETAILED DESCRIPTION OF ILLUSTRATIVE EMBODIMENTS

Provided herein are genetically engineered microbes and methods for using the genetically engineered microbes. As used herein, “genetically engineered microbe,” “genetically modified microbe,” “process control strain,” and “sample process control strain” are used interchangeably and refer to a microbe into which has been introduced an exogenous polynucleotide. A genetically modified microbe is not naturally occurring. As used herein, a “microbe” refers to a prokaryotic cell that is a member of the domain Bacteria.

As used herein, the term “polynucleotide” refers to a polymeric form of nucleotides of any length, either deoxynucleotides or ribonucleotides, and includes both double- and single-stranded DNA and RNA. As used herein, an “exogenous” polynucleotide refers to a polynucleotide that is not normally or naturally found in a microbe. An “endogenous polynucleotide” is also referred to as a “native polynucleotide.”

A microbe that is used as the basis to produce a genetically engineered microbe described herein is a microbe that is not normally shed by a human or an animal, e.g., the microbe is not expelled by a human through the genital tract or the intestinal tract. The microbe is not normally a member of the microbiota of a human or an animal. For instance, the microbe is not a pathogen of a human or an animal, and is not a member of the ecological community normally present in the gut of a human or an animal. Microbes that are pathogens of humans and animals are known to the skilled person. Likewise, microbes that are members of the gut of a human or an animal are also known to the skilled person.

A microbe that is used as the basis for producing a genetically engineered microbe is amenable to genetic manipulation. For instance, a DNA molecule can be introduced into the microbe using standard technologies such as transformation, transduction, or conjugation. In one embodiment, a genetically engineered microbe is a Gram negative microbe, and in another embodiment a genetically engineered microbe is a Gram positive microbe.

Examples of microbes having these characteristics are readily available. In one embodiment, an example of a Gram negative microbe is a Pseudogulbenkiania sp., such as P. subflava (Lin et al. 2008 Intl J. Syst. Evol. Microbiol. 58: 2384-2388), available from the Belgian Co-ordinated Collection of Microorganisms (BCCM/LMG) as strain

LMG 24211 and from the Bioresource Collection and Research Center, Taiwan (BCRC) as strain BCRC 17727. Another example of a Gram negative microbe is Pantoea stewartii subsp. stewartii strain ATCC 8199, available from the American Type Culture Collection (ATCC). In one embodiment, an example of a Gram positive microbe is a Geobacillus sp., such as G. thermodenitrificans ATCC 29492 (Manachini et al. 2000 Intl J. Syst. Evol. Microbiol. 50: 1331-1337) available from ATCC.

A genetically engineered microbe includes an exogenous polynucleotide. The exogenous polynucleotide acts as a site for hybridization of one primer that is used for amplification by a polymerase chain reaction (PCR). Thus, an exogenous polynucleotide includes a series of consecutive nucleotides having a sequence that is unique in that microbe's genome. In one embodiment, the exogenous polynucleotide is at least the length of a primer, e.g., at least 20 nucleotides. The exogenous polynucleotide can be longer, and the length of an exogenous polynucleotide is limited to what can be introduced into a microbe using genetic technologies. The length of an exogenous polynucleotide present in the genome of a genetically engineered microbe may be no greater than 500 nucleotides, no greater than 1,000 nucleotides, no greater than 1,500 nucleotides, no greater than 2,000 nucleotides, or no greater than 2,500 nucleotides. The nucleotide sequence of the exogenous polynucleotide is not intended to be limiting; any nucleotide sequence can be used. Examples of exogenous polynucleotides include, but are not limited to, a transposon and a vector such as phage or a plasmid, or a portion thereof.

In one embodiment a useful exogenous polynucleotide encodes a selectable marker. A selectable marker encodes a molecule that inactivates or otherwise detects or is detected by a compound in the growth medium. For example, a selectable marker can render the genetically engineered microbe resistant to an antibiotic, or it can confer a compound-specific metabolism on the transformed cell. Examples of selectable markers include, but are not limited to, those that confer resistance to kanamycin, ampicillin, chloramphenicol, tetracycline, neomycin, and other antibiotics.

The exogenous polynucleotide is integrated into genomic DNA of the microbe, such as the chromosome or a plasmid, provided the plasmid is stably maintained in the microbe such that each microbial cell receives at least one copy during cell division. In one embodiment, the exogenous polynucleotide is integrated into the chromosome. Since the exogenous polynucleotide acts as a site for hybridization of a primer that is used for a PCR amplification, the exogenous polynucleotide is inserted adjacent to endogenous nucleotides that can be used as a site for hybridization of a second primer for the PCR amplification. While any series of endogenous nucleotides can be used, the skilled person will recognize that unique endogenous nucleotides are preferred to promote specific hybridization and prevent non-specific hybridization with other endogenous sequences. The location of the inserted exogenous polynucleotide may be anywhere in the genome, and in one embodiment, the exogenous polynucleotide is not inserted into a gene that encodes a gene product important in growth of the microbe. Examples of nucleotides that can be used include, but are not limited to, nucleotides that encode a ribosomal component of the large or small subunit of a prokaryotic ribosome, such as 16S or 23S.

Also provided are polynucleotides that can be used as primers and probes in the methods described herein. As used herein, a “primer” refers to a type of polynucleotide that includes a sequence complementary or partially complementary to a target polynucleotide present in the genetically engineered microbe, or a microbe that may be in a sample, which hybridizes to the target polynucleotide through base pairing. After hybridization to the target polynucleotide a primer may serve as a starting-point for an amplification reaction and the synthesis of an amplification product. A “primer pair” refers to two primers that can be used together for an amplification reaction. The length of a primer is one that is useful in a PCR. The length of a primer can vary and in one embodiment is at least 15 to no greater than 40 nucleotides in length (for instance, 15, 16, 17, 18, 19, 20, 21, 22, 23, 24, 25, 26, 27, 28, 29, 30, 31, 32, 33, 34, 35, 36, 37, 38, 39, or 40 nucleotides), however, longer primers are possible. Typically, two primers are long enough to hybridize to the target polynucleotide and not hybridize to other non-target polynucleotides present in other microbes that may be present in the amplification reaction. Non-limiting examples of primers are shown in Table 1 and Table 4.

As used herein, a “target polynucleotide” refers to a polynucleotide present in the genetically engineered microbe, or a microbe that may be in a sample, which is to be detected as described herein. A “target polynucleotide” may be a natural polynucleotide, e.g., a nucleotide sequence naturally existing in a microbe, a recombinant polynucleotide, e.g., an exogenous nucleotide sequence in a microbe that is the result human intervention, or a combination thereof. A target polynucleotide may be genomic DNA, plasmid DNA, or an amplified product that is the result of a PCR amplification.

As used herein, the term “complement” refers to the ability of two single stranded polynucleotides to base pair with each other, where an adenine on one polynucleotide will base pair to a thymine on a second polynucleotide and a cytosine on one polynucleotide will base pair to a guanine on a second polynucleotide. Two polynucleotides are complementary to each other when a nucleotide sequence in one polynucleotide can base pair with a nucleotide sequence in a second polynucleotide. As used herein, “hybridizes,” “hybridizing,” and “hybridization” means that a single stranded polynucleotide forms a noncovalent interaction with a complementary polynucleotide or partially complementary polynucleotide under certain conditions. Hybridization is affected by many factors including the degree of complementarity between two polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands. Hybridization conditions useful in a PCR are described herein.

As used herein, a “probe” is a polynucleotide that is complementary to at least a portion of an amplification product formed using two primers. Typically, a probe is long enough to hybridize to the target polynucleotide (and the amplification product) and not hybridize to other non-target polynucleotides present in other microbes that may be present in the amplification reaction. Probe lengths are generally at least 15 nucleotides to no greater than 40 nucleotides in length (for instance, 15, 16, 17, 18, 19, 20, 21, 22, 23, 24, 25, 26, 27, 28, 29, 30, 31, 32, 33, 34, 35, 36, 37, 38, 39, or 40 nucleotides). In one embodiment, a probe and the primers with which the probe is used will not hybridize to the same nucleotides of an amplification product. A probe will hybridize to one strand of a target polynucleotide and to one strand of an amplified product, and is typically designed to hybridize to the amplified product before the primer that hybridizes to that strand. Non-limiting examples of primers are shown in Table 1 and Table 4.

A probe may further include additional nucleotides. Such additional nucleotides may be present at either the 5′ end, the 3′ end, or both, and include, for instance, nucleotides that form a hairpin loop, and other nucleotides that permit the probe to be used as, for instance, a molecular beacon.

Nucleotides of a primer or a probe may be modified. Such modifications can be useful to increase stability of the polynucleotide in certain environments. Modifications can include a nucleic acid backbone, base, sugar, or any combination thereof. The modifications can be synthetic, naturally occurring, or non-naturally occurring. A primer or probe can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide nucleic acids (Nielson et al., U.S. Pat. No. 5,539,082; Egholm et al., Nature, 1993, 365:566-568). Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-O-trifluoromethyl nucleotides, 2′-O-ethyl-trifluoromethoxy nucleotides, 2′-O-difluoromethoxy-ethoxy nucleotides, or 2′-deoxy nucleotides.

Primers and probes may include a label. A “label” refers to a moiety attached (covalently or non-covalently), or capable of being attached, to an primer or probe, which provides or is capable of providing information about the primer or probe (e.g., descriptive or identifying information about the polynucleotide) or another polynucleotide with which the labeled primer or probe interacts (e.g., hybridizes). Labels can be used to provide a detectable (and optionally quantifiable) signal. Exemplary labels include, but are not limited to, fluorophore labels (including, e.g., quenchers or absorbers), non-fluorescent labels, colorimetric labels, chemiluminescent labels, bioluminescent labels, radioactive labels, mass-modifying groups, affinity labels, magnetic particles, antigens, enzymes (including, e.g., peroxidase, phosphatase), substrates, and the like. Labels may provide signals detectable by fluorescence, radioactivity, colorimetry, X-ray diffraction or absorption, magnetism, enzymatic activity, and the like. Affinity labels provide for a specific interaction with another molecule. Examples of affinity labels include, for instance, biotin, avidin, streptavidin, dinitrophenyl, digoxigenin, cholesterol, polyethyleneoxy, haptens, and peptides such as antibodies.

In certain aspects a label is a fluorophore. A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength. Fluorophore labels include, but are not limited to, dyes of the fluorescein family, the carboxyrhodamine family, the cyanine family, and the rhodamine family. Other families of dyes that can be used in the invention include, e.g., polyhalofluorescein-family dyes, hexachlorofluorescein-family dyes, coumarin-family dyes, oxazine-family dyes, thiazine-family dyes, squaraine-family dyes, chelated lanthanide-family dyes, the family of dyes available under the trade designation Alexa Fluor™, from Molecular Probes, and the family of dyes available under the trade designation Bodipy™, from Invitrogen (Carlsbad, Calif.). Dyes of the fluorescein family include, e.g., 6-carboxyfluorescein (FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), 2′-chloro-7′-phenyl-1,4-dichloro-6-carboxyfluorescein (VIC), 6-carboxy-X-rhodamine (ROX), and 2′,4′,5′,7′-tetrachloro-5-carboxy-fluorescein (ZOE). Dyes of the carboxyrhodamine family include tetramethyl-6-carboxyrhodamine (TAMRA), tetrapropano-6-carboxyrhodamine (ROX), Texas Red, R110, and R6G. Dyes of the cyanine family include Cy2, Cy3, Cy3.5, Cy5, Cy5.5, and Cy7. Fluorophores are readily available commercially from, for instance, Perkin-Elmer (Foster City, Calif.), Molecular Probes, Inc. (Eugene, Oreg.), and Amersham GE Healthcare (Piscataway, N.J.).

The label may be a quencher. The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a fluorophore (a fluorescent quencher). This phenomenon is generally known as fluorescent resonance energy transfer (FRET). Alternatively, a quencher can dissipate the energy absorbed from a fluorophore as heat (a non-fluorescent quencher). Quenchers may be fluorescent quenchers or non-fluorescent quenchers. Fluorescent quenchers include, but are not limited to, TAMRA, DABCYL, DABSYL, cyanine dyes including nitrothiazole blue (NTB), anthraquinone, malachite green, nitrothiazole, and nitroimidazole compounds. Exemplary non-fluorescent quenchers that dissipate energy absorbed from a fluorophore include those available under the trade designation Black Hole™, from Biosearch Technologies, Inc. (Novato, Calif.), those available under the trade designation Eclipse Dark™, from Epoch Biosciences (Bothell, Wash.), those available under the trade designation Qx1™, from Anaspec, Inc. (San Jose, Calif.), and those available under the trade designation Iowa Black™, from Integrated DNA Technologies (Coralville, Iowa).

Typically, a fluorophore and a quencher are used together, and may be on the same or different primer or probe. When paired together, a fluorophore and fluorescent quencher can be referred to as a donor fluorophore and acceptor fluorophore, respectively. A number of convenient fluorophore/quencher pairs are known in the art (see, for example, Glazer et al, Current Opinion in Biotechnology, 1997;8:94-102; Tyagi et al., 1998, Nat. Biotechnol., 16:49-53) and are readily available commercially from, for instance, Molecular Probes (Junction City, Oreg.), and Applied Biosystems (Foster City, Calif.). Examples of donor fluorophores that can be used with various acceptor fluorophores include, but are not limited to, fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2-,2′-disulfonic acid derivatives. Acceptor fluorophores typically depend upon the donor fluorophore used. Examples of acceptor fluorophores include, but are not limited to, LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorophores are readily available commercially from, for instance, Molecular Probes or Sigma Chemical Co. (St. Louis, Mo.).

Examples of probes useful in real-time assays using donor and acceptor fluorophores include, but are not limited to, adjacent probes (Cardullo et al., 1988, Proc. Natl. Acad. Sci. USA, 85:8790-8794; Wittwer, 1997, BioTechniques, 22:130-131), and Taqman probes (Holland et al., 1991, Proc. Natl. Acad. Sci. USA, 88:7276-7280; Livak et al., 1995, PCR Methods Appl., 4:357-62). Examples of probes and primers useful in real-time assays using fluorphores and non-fluorescent quenchers include, but are not limited to, molecular beacons (Tyagi et al., 1996, Nat. Biotechnol., 14:303-308; Johansson et al., 2002, J. Am. Chem. Soc., 124:6950-6956), scorpion primers (including duplex scorpion primers) (Whitcombe et al., U.S. Pat. No. 6,326,145; Whitcombe et al., 1999, Nat. Biotechnol., 17:804-817), amplifluor primers (Nazarenko et al., 1997, Proc. Natl. Acad. Sci. USA, 25:2516-2521), and light-up probes (Svanvik et al., 2000, Anal. Biochem., 287:179-182).

Primers and probes can be produced in vitro or in vivo. For instance, methods for in vitro synthesis include, but are not limited to, chemical synthesis with a conventional DNA/RNA synthesizer. Commercial suppliers of synthetic polynucleotides and reagents for such syntheses are well known. Methods for in vitro synthesis also include, for instance, in vitro transcription using a circular or linear expression vector in a cell free system. Expression vectors can also be used to produce a polynucleotide of the present invention in a cell, and the polynucleotide then isolated from the cell.

Primers useful in the methods described herein can be designed using readily available computer programs, such as Primer 3 (Thermo Fisher Scientific), Primer Express® (Applied Biosystems, Foster City, Calif.), and IDT® OligoAnalyzer 3.0 (Integrated DNA Technologies, Coralville, Iowa). Factors that can be considered in designing primers include, but are not limited to, melting temperatures, primer length, size of the amplification product, and specificity. Primers useful in the amplification methods described herein typically have a melting temperature (T_{M}) that is greater than at least 55° C., at least 56° C., at least 57° C., at least 58° C., at least 59° C., at least 60° C., at least 61° C., at least 62° C., at least 63° C., or at least 64° C. The T_{M} of a primer can be determined by the Wallace Rule (Wallace et al., 1979, Nucleic Acids Res., 6:3543-3557) or by readily available computer programs, such as IDT Oligo Analyzer 3.0. In one embodiment, the primers of a primer pair will have T_{M}s that vary by no greater than 5° C., no greater than 4° C., no greater than 3° C., no greater than 2° C., or no greater than 1° C.

Designing a probe can be done in a manner similar to designing the primers described herein. Factors that can be considered in designing probes useful in the methods described herein include, but are not limited to, melting temperature, length, and location of the probe with respect to the primers. Typically, a probe will have a T_{M} that is greater than or equal to the highest T_{M} of the primers with which the probe is to be used. Preferably, a probe has a T_{M} that is at least 1° C. greater, at least 2° C. greater, at least 3° C. greater, at least 4° C. greater, at least 5° C. greater, at least 6° C. greater, at least 7° C. greater, or at least 8° C. greater than the highest T_{M} of the primer pair with which the probe is to be used. Typically, the greater Tm permits the probe to hybridize before the primer, which aids in maximizing the labeling of each amplification product with probe.

A genetically engineered microbe described herein is useful as a microbe-based biosensor to aid in determining the concentration or one or more microbes in a sample. Typically, a sample is analyzed for at least two microbes, the genetically engineered microbe and a test microbe. A test microbe can be any microbe whose presence is suspected in a sample. In one embodiment, the test microbe can be a pathogen associated with disease in a human or a non-human animal. Examples of such microbes include, but are not limited to, Campylobacter spp. (such as C. jejuni and C. lari), Listeria monocytogenes, Salmonella spp., Shigella spp., Clostridium perfringens, Legionella pneumophila, Listeria monocytogenes, Vibrio spp. (such as V. cholera and V. paraheamolyticus, and E. coli (such as E. coli O157:H7, a Shiga-toxin producing E. coli, or an enteropathogenic (eaeA-positive) E. coli). In one embodiment, the test microbe can be a microbe indicative of fecal contamination of a sample. A test microbe may be referred to as a microbial contaminant. Examples of such microbes include, but are not limited to, bacteria originating in the intestines of humans and/or animals such as fecal coliform microbes, members of the genus Bacteroides (Bacteroides spp.), and members of the genus Enterococcus (Enterococcus spp.).

In one embodiment, a method provided herein includes adding to a sample a known number of a genetically engineered microbe, and extracting polynucleotides of the microbes present in the sample. A sample may be from an environmental, biological, or industrial source. Examples of a sample source include, but are not limited to, recreational water, such as ocean water, pond water, lake water, creek water, river water, and water from a swimming pool, a hot tub, and a sauna. Other sample sources include, but are not limited to, groundwater, leachate, wastewater, sewer water, blackwater, graywater, bilge water, ballast water, feed water, process water, industrial water, irrigation water, rain water, runoff water, cooling water, nonpotable water, potable water, and drinking water. Other environmental samples include, but are not limited to, beach sands, sediments, and soils. Biological samples include, but are not limited to samples from aquatic organisms such as shellfish, and terrestrial organisms. Other biological samples include clinical specimens, such as tissue, stool, or a body fluid such as cerebrospinal fluid, blood, urine, sputum, and synovial fluid. Another type of sample is a food sample, such as meat, fish, milk, cheese, fruit and vegetable.

A sample may optionally be processed to prepare it for extraction of polynucleotides. In one embodiment a sample is concentrated. Methods for concentrating a sample to increase the concentration of any microbes present are known to the skilled person and are routine. Examples include, but are not limited to, filtration, centrifugation, flow cytometric cell sorting, and antibody-based cell capturing.

The genetically engineered microbe is added to the sample either before or after the optional concentrating. In one embodiment, the number of the genetically engineered microbe added can be at least 10 cells per liter of sample (cells/L), at least 100 cells/L, at least 1,000 cells/L, at least 1×10⁴ cells/L, at least 1×10⁵ cells/L, at least 1×10⁶ cells/L, at least 1×10⁷ cells/L, or at least1×10⁸ cells/L. In one embodiment, the number of the genetically engineered microbe added can be no greater than 1×10⁸ cells/L, no greater than 1×10⁷ cells/L, no greater than 1×10⁶ cells/L, no greater than 1×10⁵cells/L, no greater than 1×10⁴ cells/L, no treater than 1,000 cells/L, or no greater than 100 cells/L. In one embodiment, when the sample is a solid, such as a fecal sample or a food sample, the units can be cells per gram instead of cells per liter.

In one embodiment, a standard curve is generated for use in quantifying the amount of the genetically engineered microbe added to a sample. As described in Examples 2 and 4, a standard curve can be generated by plotting the results of a quantitative PCR as the threshold cycle (C_{T}) as a function of the quantity of template DNA added (copies/μL).

Extraction of polynucleotides present in the microbes in the sample, including the genetically engineered microbe, can be accomplished using techniques known to the skilled person and routine in the art. Extraction includes the physical disruption of the membranes of the cells. Examples include, for instance, boiling, hydrolysis with proteinases, exposure to ultrasonic waves, detergents, strong bases, organic solvents such as phenol chloroform, glass bead milling, or glass milk adsorption. Commercially available kits can be used in the methods described herein, such as, PowerSoil DNA Isolation Kit (MoBio), FastDNA Spin Kit for Soil DNA Extraction (MP Biomedicals, and QIAamp DNA Stool Mini Kit (Qiagen).

The polynucleotides used as targets in the methods may be of any molecular weight and in single-stranded form, double-stranded form, circular, linear, plasmid, etc. Various types of polynucleotides can be separated from each other (e.g., RNA from DNA, or double-stranded DNA from single-stranded DNA). For example, polynucleotides of at least 100 bases in length, longer molecules of 1,000 bases to 10,000 bases in length, and even high molecular weight nucleic acids of up to about 4.3 megabases can be used in the methods described herein.

The method also includes adding primers and optional probes to the sample and using PCR to amplify target polynucleotides present in test microbe. In one embodiment, the extracted DNA is divided into multiple aliquots and different primers and optional probes are added to each aliquot to specifically amplify DNA from different test microbes. For instance, when a C. jejuni and the genetically engineered microbe are being detected the sample is divided into at least two aliquots and primers specific for C. jejuni are added to one aliquot and primers specific for the genetically engineered microbe are added to the other aliquot.

The resulting amplified target polynucleotides are detected and used to determine the DNA extraction efficiency (also referred to as DNA recovery efficiency) of the genetically engineered microbe. Briefly, the DNA concentration of the genetically engineered microbe in the sample can be determined using the results of the PCR reaction. Since the target polynucleotide amplified by the PCR is present one per genome, this can be converted from copies/μL to cells/μL. This can be compared to the amount of the genetically engineered microbe actually added to determine the DNA extraction efficiency. The DNA extraction efficiency can then be used to normalize the concentration of the test microbe in the sample.

Optionally, before the PCR to amplify target polynucleotides a preliminary amplification reaction, referred to as a specific target amplification, may be done. Specific target amplification can be used to increase the amount of DNA template for the PCR. The specific target amplification is a limited cycle amplification, for instance, 12 to 16 cycles, using all the primers that will be used in the final PCR (see, for instance, Spurgeon et al., 2008, PLoS ONE, 3:e1662).

Primer sets and probes useful for the specific detection of many different fecal indicator microbes and pathogens using a PCR-based method are known to the skilled person, see for instance, Ishii et al., 2013, Applied Environmental Microbiology 79: 2891-2898). Kits are also commercially available for detecting specific microbes in environmental samples, such as Microbial DNA qPCR Assay Kits (Qiagen) and Real Time PCR Pathogen Detection Kit (Primerdesign, Southampton, UK).

The primers and optional probes are used in a PCR to amplify a specific DNA in a sample. Numerous different PCR methods are known to the person skilled in the art, and can be directly applied or adapted for use with the methods described herein for quantification of microbes in a sample. Generally, the amplification is based on repeated cycles of the following basic steps: denaturation of double-stranded polynucleotides, followed by primer annealing to the target polynucleotide, and primer extension by a polymerase. The primers are designed to anneal to opposite strands of the DNA, and are positioned so that the polymerase-catalyzed extension product of one primer can serve as the template strand for the other primer. The amplification process can result in the exponential increase of discrete polynucleotide fragments whose length is defined by the 5′ ends of the primers.

Generally, these steps are achieved in a cycling step. A typical cycling step used in DNA amplification involves two target temperatures to result in denaturation, annealing, and extension. The first temperature is an increase to a predetermined target denaturation temperature high enough to separate the double-stranded target polynucleotide into single strands. Generally, the target denaturation temperature of a cycling step is approximately 92° C. to 98° C., such as 94° C. to 96° C., and the reaction is held at this temperature for a time period ranging between 0 seconds to 5 minutes. The temperature of the reaction mixture is then lowered to a second target temperature. This second target temperature allows the primers (and probe(s), if present) to anneal or hybridize to the single strands of DNA, and promote the synthesis of extension products by a DNA polymerase. Generally, the second temperature of a cycling step is approximately 57° C. to 63° C., such as 59° C. to 61° C., and the reaction is held at this temperature for a time period ranging between 0 seconds to 1 minute. This second temperature can vary greatly depending upon the primers (and probe(s), if present) and target polynucleotide used. This completes one cycling step. The next cycle then starts by raising the temperature of the reaction mixture to the denaturation temperature. Typically, the cycle is repeated to provide the desired result, which may be to produce a quantity of DNA and/or detect an amplified product. For use in detection, the number of cycling steps will depend on the nature of the sample. For instance, if the sample is a complex mixture of polynucleotides, more cycling steps may be required to amplify the target polynucleotide sufficient for detection. Generally, the cycling steps are repeated at least 14 times, but may be repeated as many as 40, 60, or even 100 times. As will be understood by the skilled artisan, the above description of the thermal cycling reaction is provided for illustration only, and accordingly, the temperatures, times and cycle number can vary depending upon the nature of the thermal cycling reaction and application.

Optionally, a third temperature is also used in a cycling step. The use of three target temperatures also results in denaturation, annealing, and extension, but separate target temperatures are used for the denaturation, annealing, and extension. When three target temperatures are used the annealing temperatures generally range from 45° C. to 72° C., depending upon the application. The third target temperature is for extension, is typically held for a time period ranging between 30 seconds to 10 minutes, and occurs at a temperature range between the annealing and denaturing temperatures.

DNA polymerases for use in the methods and compositions of the present invention are capable of effecting extension of a primer according to the methods of the present invention. Accordingly, a preferred polymerase is one that is capable of extending a primer along a target polynucleotide. Preferably, a polymerase is thermostable. A thermostable polymerase is a polymerase that is heat stable, i.e., the polymerase catalyzes the formation of primer extension products complementary to a template and does not irreversibly denature when subjected to the elevated temperatures for the time necessary to effect denaturation of double-stranded template nucleic acids. Useful thermostable polymerases are well known and used routinely. Thermostable polymerases have been isolated from Thermus flavus, T ruber, T thermophilus, T aquaticus, T lacteus, T rubens, Bacillus stearothermophilus, and Methanothermus fervidus.

A polymerase typically initiates synthesis at the 3′-end of a primer annealed to a target polynucleotide, and proceeds in the 5′-direction along the target polynucleotide. A polymerase may possess a 5′ to 3′ exonuclease activity, and hydrolyze intervening, annealed probe(s), if present, to release portions of the probe(s), until synthesis terminates. Examples of suitable polymerases having a 5′ to 3′ exonuclease activity include, for example, Tfi, Taq, and FastStart Taq. In other aspects, the polymerase has little or no 5′ to 3′ exonuclease activity so as to minimize degradation of primer, termination or primer extension polynucleotides. This exonuclease activity may be dependent on factors such as pH, salt concentration, whether the target is double stranded or single stranded, and so forth, all of which are familiar to one skilled in the art. Examples of suitable polymerases having little or no 5′ to 3′ exonuclease activity include Klentaq (Sigma, St. Louis, Mo.).

The presence or absence of an amplified product can be determined or its amount measured. Detecting an amplified product can be conducted by standard methods well known in the art and used routinely. The detecting may occur, for instance, after multiple amplification cycles have been run, or during each amplification cycle (typically referred to as quantitative PCR or realtime PCR). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. The most convenient label is typically fluorescent, which may be used in various formats including, but are not limited to, the use of donor fluorophore labels, acceptor fluorophore labels, flourophores, quenchers, and combinations thereof.

Quantitative PCR (qPCR) (also referred as real-time PCR) is more useful under some circumstances because it provides not only a quantitative measurement, but also reduced time and risk of contamination. qPCR is the direct monitoring of the progress of a PCR amplification as it is occurring without the need for repeated sampling of the reaction products. In qPCR, production of the amplified product can be monitored using a signaling mechanism (e.g., fluorescence) as it is generated and is tracked after the signal rises above a background level but before the reaction reaches a plateau. The number of cycles required to achieve a detectable or threshold level of fluorescence varies directly with the concentration of amplifiable targets at the beginning of the PCR process. This relationship enables a measure of signal intensity to provide a measure of the amount of target polynucleotide in a sample in real time.

The types of assays using the various formats may include the use of one or more primers that are labeled (for instance, scorpions primers, amplifluor primers), one or more probes that are labeled (for instance, adjacent probes, Taqman probes, light-up probes, molecular beacons), or a combination thereof. The skilled person will understand that in addition to these known formats, new types of formats are routinely disclosed. The methods described herein are not limited by the type of method or the types of probes and/or primers used to detect an amplified product.

It is understood that the methods described herein are not limited by the device used to conduct the amplification and detection of the amplified product. For example, suitable devices may include conventional amplification devices such as, for instance, those available from Bio-Rad, Thermo Fisher Scientific, and Beckman. It may be preferred that the method is practiced in connection with a microfluidic device. “Microfluidic” refers to a device with one or more fluid passages, chambers, or conduits that have at least one internal cross-sectional dimension, e.g., depth, width, length, diameter, etc., that is less than 500 μm, and typically between 0.1 μm and 500 μm. Typically, a microfluidic device includes a plurality of chambers (e.g., amplification reaction chambers, loading chambers, and the like), each of the chambers defining a volume for containing a sample. Some examples of potentially suitable microfluidic devices include those available from Fluidigm.

Also provided is a kit for practicing a method described herein. In one embodiment, the kit includes a genetically engineered microbe and two primers for the PCR amplification of a target polynucleotide of the genetically engineered microbe. Optionally, the kit also includes a probe. The primers and optional probe are in a suitable packaging material in an amount sufficient for at least one assay. Other reagents needed to practice the method are also included. Reagents useful for extraction of DNA from cells present in a sample, including the genetically engineered microbe and one or more other microbes can be included. Reagents useful for PCR amplification, including a polymerase, a buffer solution (either prepared or present in its constituent components, where one or more of the components may be premixed or all of the components may be separate), and the like, can be included. Yet other reagents, such as a medium for growth of the genetically engineered microbe, can be included. Instructions for use of the contents of the kit are also typically included. A kit may also include a container for the PCR reaction, such as a microfluidic chip.

As used herein, the phrase “packaging material” refers to one or more physical structures used to house the contents of the kit. The packaging material is constructed by known methods, preferably to provide a sterile, contaminant-free environment. The packaging material has a label which indicates that the kit can be used to estimate the number of microbes in a sample, such as an environmental sample. In addition, the packaging material contains instructions indicating how the materials within the kit are employed to extract DNA, amplify DNA, calculate the DNA extraction efficiency of the genetically engineered microbe, estimate the concentration of a microbe in a sample, or a combination thereof. As used herein, the term “package” refers to a solid matrix or material such as glass, plastic, paper, foil, and the like, capable of holding within fixed limits a component of the kit. Thus, for example, a package can be a plastic vial used to contain appropriate quantities of a primer pair. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of and/or “consisting essentially of are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Also, in the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

Various bacteria, viral, and protozoan pathogens can cause human disease. Rapid identification of disease-causing agents in human patients (e.g., stool samples) is important for the appropriate treatment of patients and to prevent the spread of diseases (Ishii et al., 2013, Applied and Environmental Microbiology 79: 2891-2898). Occurrences of pathogens in food, water, and other environmental matrices are also potential threats to human health. To prevent disease outbreaks, levels of pathogen contamination and their potential risks to human health should be properly assessed (Ishii et al., 2014, Environmental Science & Technology 48: 4744-4749). Currently, fecal indicator bacteria such as Escherichia coli and enterococci are used to monitor levels of fecal contamination in waterways. However, a growing number of reports suggest the environmental survival and growth of fecal indicator bacteria (FIB), which confound the use of these bacteria as indicators of fecal contamination (Byappanahalli et al., 2012, Microbiology and Molecular Biology Reviews 76: 685-706, Ishii and Sadowsky, 2008, Microbes and Environments 23: 101-108). In addition, poor correlation was sometimes observed between concentrations of FIB and pathogens (Ishii et al., 2014, Environmental Science & Technology 48: 4744-4749).

Molecular tools, such as polymerase chain reaction (PCR) and its derivative quantitative PCR (qPCR) have been frequently used to detect and quantify pathogenic bacteria in various samples. In addition, various methods have been developed to simultaneously detect multiple pathogens (Kronlein et al., 2014, Water Environment Research 86: 882-897). Among those, microfluidic qPCR (MFQPCR)-based approach is promising because it can provide quantitative information of multiple pathogens for many samples in a timely manner (Ishii et al 2013, Ishii et al 2014a). In a MFQPCR chip, different qPCRs can be run simultaneously in nanoliter (10⁻¹² liter)-volume chambers that are present in high densities on the chip. The MFQPCR has been used to quantify multiple pathogens in water, sand, sediment, and algae samples (Byappanahalli et al., 2015, Environmental Science & Technology Letters 2: 347-351; Zhang et al., 2016, Science of the Total Environment 573, 826-830). Quantitative information obtained by MFQPCR approach can be used for risk assessment (Ishii et al., 2014, Environmental Science & Technology 48: 4744-4749). PCR-based approaches including MFQPCR are generally sensitive to detect target molecules present at low concentrations. However, the reaction can be inhibited by PCR-inhibitors such as humic substances, which are commonly present in soil and sediment samples. With the presence of PCR inhibitors, inaccurate quantitative results can be obtained by qPCR. Therefore, it is helpful to prepare DNA samples in high purity. During the DNA extraction and purification procedure, some portion of DNA can be lost. In addition, when microbial cells from water samples are concentrated, for example, by membrane filtration technique, some portion of microbial cells can be lost as well. Therefore, DNA recovery efficiency calculated as the proportion of the target DNA molecules before and after sample processing (e.g., water filtration and DNA extraction) can become lower than 100%. However, most currently-available PCR-based pathogen quantification methods assume that the DNA recovery efficiency is 100%. This underestimates the quantity of pathogens, and therefore, can bring inaccurate risk estimates.

Use of a sample process control (SPC) was proposed to assess DNA recovery efficiency. In order to obtain DNA recovery efficiencies, a known amount of SPC is added to the samples before sample processing (e.g., water filtration and DNA extraction). After DNA extraction, the quantity of the SPC is quantified by SPC-specific qPCR. By comparing the quantity of SPC inoculated and the quantity of SPC measured by qPCR, the DNA recovery efficiency can be calculated. The DNA recovery efficiency values can be used to standardize concentrations of the target DNA molecules (e.g., pathogen marker genes). Ideally, SPC should (1) be absent when no SPC is spiked, (2) be concentrated similar to the target cells, (3) be lysed with similar effectiveness to the target cells, and (4) contain DNA that is extracted and quantified with similar efficiency to that of the target cells (Stoeckel et al 2009).

Previously, Stoeckel et al. (Stoeckel et al., 2009, Water Research 43: 4820-4827) used a plant pathogen Pantoea stewartii as a SPC strain. However, background population of Pantoea stewartii may cause inaccurate DNA recovery efficiency. Stoeckel et al. also used E. coli carrying plasmid-borne target gene. There is no background population of this genetically-engineered E. coli strain. However, the number of plasmids per cell can change by physiological state of the cells. In addition, DNA extraction efficiencies can be different between genomic DNA and plasmid DNA. Kobayashi et al. (Kobayashi et al., 2013, Applied Microbiology and Biotechnology 97: 9165-9173) used a genetically-engineered E. coli strain that has kanamycin resistant gene in its β-galactosidase gene (lacZ) as a SPC strain. They designed SPC-specific qPCR assay targeting a junction region between lacZ and kanamycin resistance gene. Because their gene modification was made on the E. coli chromosome, target gene molecule is present per genome (=per cell). However, if this strain is used to spike, it is not possible to reliably quantify E. coli levels in the environment.

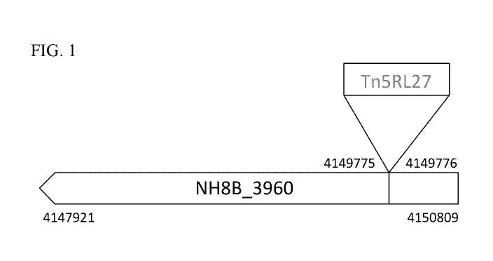

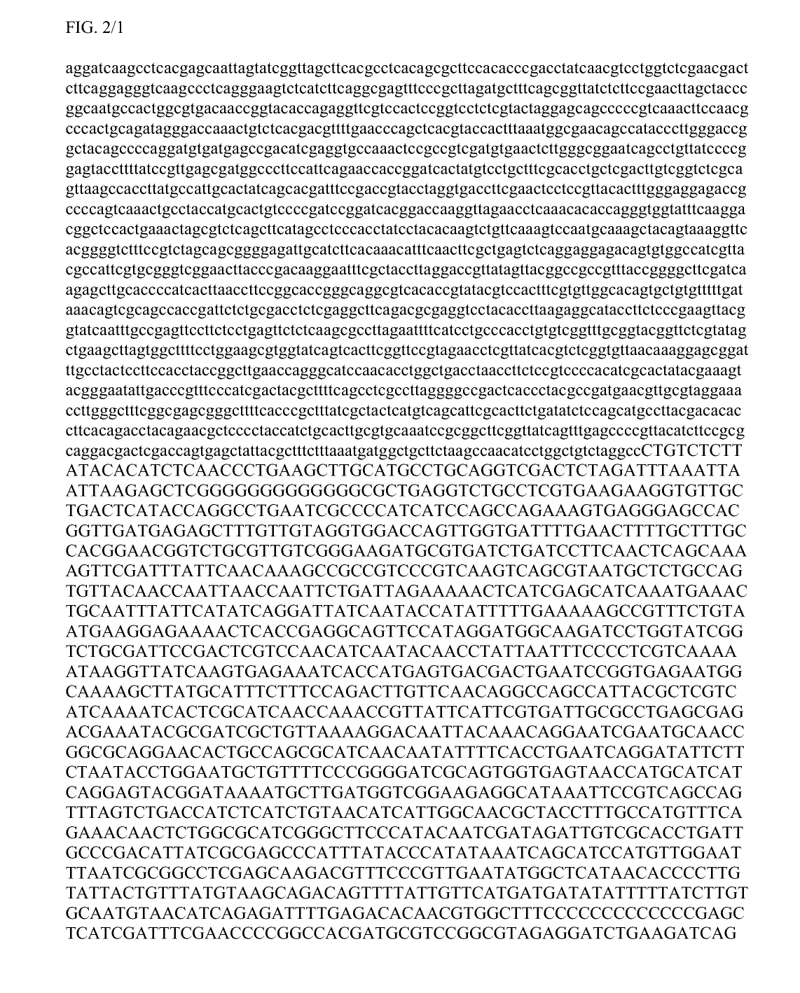

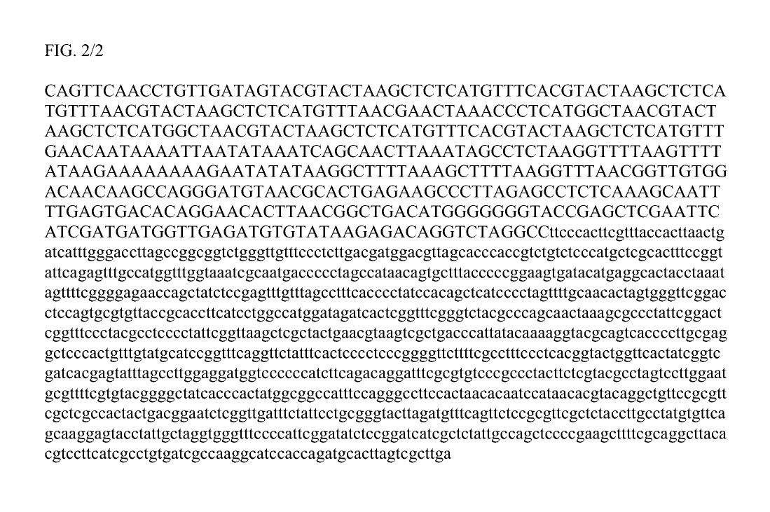

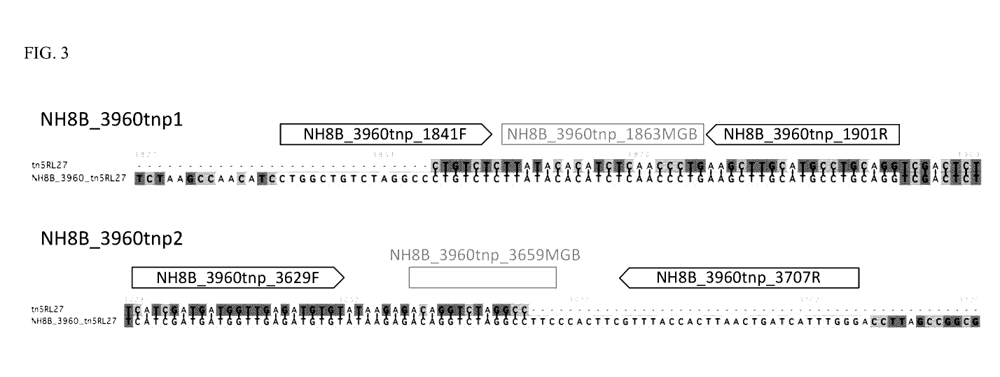

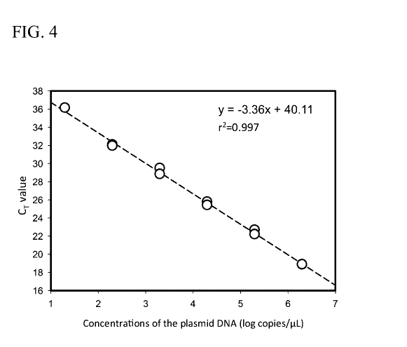

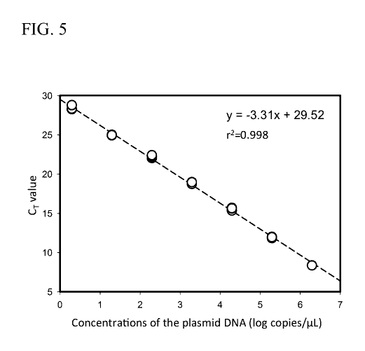

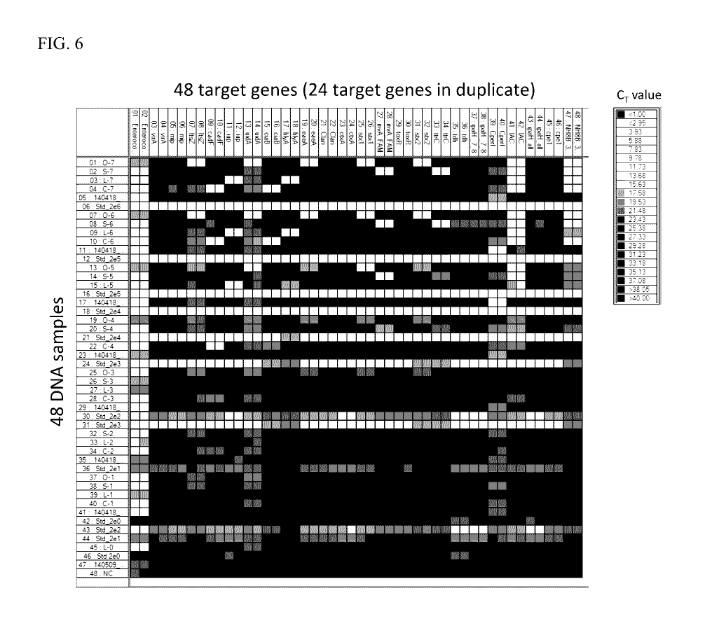

A genetically modified bacteria strain Pseudogulbenkiania sp. NH8B-1D2 has been developed as a SPC strain. Strain NH8B-1D2 has a Tn5RL27 insertion in one of its 23S rRNA gene (FIG. 1 and FIG. 2). The TaqMan-probe based qPCR assays are designed to amplify the junction regions between Tn5RL27 and the 23S rRNA gene sequences (FIG. 3). These assays can specifically quantify strain NH8B-1D2 in high efficiency (FIG. 4 and FIG. 5). No background signal was observed. This assay can be run in a microfluidic qPCR format together with the assays to quantify enteric pathogens (Ishii et al., 2013, Applied and Environmental Microbiology 79: 2891-2898); therefore, these genes can be quantified simultaneously (FIG. 6). The DNA extraction efficiency of strain NH8B-1D2 was similar to those of several enteric pathogens (E. coli O157:H7, Salmonella, Campylobacter, and Listeria). (Table 6). By using the DNA extraction efficiency of the SPC strain, the concentrations of pathogen marker genes in environmental samples can be standardized.

3-1. Strain NH8B-1D2 belongs to Proteobacteria similar to many human pathogens, but this strain itself is not considered as a human pathogen. This strain has a stable marker gene in its chromosome at one copy per genome, which can be specifically quantified by qPCR. This strain is easy to maintain in a laboratory condition. Addition of this strain does not interfere the quantification of the target bacteria pathogens (e.g., E. coli). This strain fulfills all requirements for SPC strain as listed above.

### Example 1

Example 1. Generation of mutant strains for sample process control

This example shows how Pseudogulbenkiania sp. NH8B-1D2 was created. This procedure can be applied to other bacteria. In addition, other gene knockout procedures can be used to create mutant strains that can be used for sample process control (SPC) to standardize DNA recovery efficiencies.
1-1. Pseudogulbenkiania sp. strain NH8B was previously isolated from a rice paddy soil in Niigata, Japan (Tago et al 2011). This bacterium belongs to Proteobacteria similar to many human pathogens, but this strain itself is not considered as a human pathogen. Complete genome of this strain was previously identified (Ishii et al 2011). Bacterial cells were maintained in R2A broth medium (Teknova) at 30° C.
1-2. For transposon mutagenesis, a plasmid pRL27 was used, which contains a hyperactive Tn5 transposase gene and aph gene that encodes for kanamycin resistance gene as well as R6K DNA replication origin (oriR6K)(Larsen et al 2002). E. coli WM3064, a 2,6-diaminopimelic acid (DAP) auxotroph, was used as a donor for pRL27 (Saltikov and Newman 2003). This strain was maintained in

LB agar medium supplemented with DAP (300 μg/ml) and kanamycin (100 μg/ml).
1-3. For bacteria conjugation, E. coli pRL27 donor strain WM3064 and the recipient Pseudogulbenkiania sp. strain NH8B were mixed at 1:4 ratio, and spotted on R2A agar supplemented with DAP (300 μg/ml). After overnight incubation, the cells were suspended in R2A broth and spread onto R2A agar supplemented with kanamycin (100 μg/ml). Colonies grown on this agar can grow without addition of DAP (i.e., they are not E. coli WM3064) and are resistant to kanamycin (i.e., they are not NH8B wild type strain). Because oriR6K requires π protein, encoded by pir gene, pRL27 are not stable in a host that lacks pir (e.g., NH8B strain). Therefore, colonies obtained by this conjugation experiment should have insertion of Tn5RL27 in their genome.
1-4. To recover the DNA fragment with Tn5RL27 insertion, self-ligation was performed followed by transformation. In brief, genomic DNA was extracted from the Pseudogulbenkiania sp. mutant strain NH8B-1D2. The DNA was digested with Nco I (Takarabio) and self-ligated using DNA Ligation Kit ver. 1 (Takarabio) according to the manufacturer's instruction. The self-ligated plasmid was transformed into OneShot PIR1 competent E. coli (Thermo Fisher Scientific), which possesses pir to support the replication of plasmid with oriR6K. Colonies growing in LB agar medium supplemented with kanamycin (100 μg/ml) were selected and re-grow in LB broth medium (5 mL) supplemented with kanamycin (100 μg/ml). Plasmid DNA was extracted from the colony by using QIAprep SPIN Mini Kit (Qiagen). This plasmid was named pRL27-NH8B-1D2-NcoI-1.
1-5. Location of the Tn5RL27 insertion was identified by PCR followed by sequencing as described elsewhere (Larsen et al 2002). As a result, it was identified that the TN5-RL27 was inserted in one of the eight 23 rRNA gene (locus tag NH8B_3960) (FIG. 1). The whole sequence of pRL27-NH8B-1D2-NcoI-1 is shown in FIG. 2.

### Example 2. Development of the Quantitative PCR Assay for the Mutant

This example shows the design of quantitative PCR (qPCR) assays for specific quantification of the mutant strain NH8B-1D2. The quantitative performance of the assay was tested with plasmid DNA (pRL27-NH8B-1D2-NcoI-1).
2-1. Specific qPCR assays with TaqMan probes were designed using Primer Express software (Thermo Fisher Scientific). To increase the specificity, the junction region between Tn5-RL27 and NH8B_3960 sequences was targeted (FIG. 3). Two qPCR assays were designed (Table 1).
2-2. Prior to the qPCR, linearized plasmid DNA solution was prepared. The pRL27-NH8B-1D2-NcoI-1 was digested by Nco I according to the manufacturer's instructions.
2-3. The linearized plasmid was purified using FastGene Gel/PCR Extraction Kit (Nippongene), and quantified using PicoGreen dsDNA quantification reagent (Thermo Scientific).
2-4. The linearized plasmid solution was serially diluted (2×10¹−2×10⁷ copies/μL) with nuclease-free water, and stored at −20° C.
2-5. The qPCR master mix was prepared according to Table 2, and aliquots (9 μL) were dispensed into 96-well PCR plate.
2-6. The serial dilution of the linearized plasmid DNA (1 μL) was added to each well of the PCR plate and mixed well.
2-7. The qPCR was performed using StepOnePlus Real-Time PCR system (Thermo Scientific) in duplicate with the following conditions: 95° C. for 10 min at initial annealing step, following with 40 cycles at 95° C. for 10 sec, and 60° C. for 30 sec. Fluorescence signals (FAM and ROX) were read at 60° C. The FAM fluorescence signals come from the breakdown of the TaqMan probe; therefore, the FAM signal intensity should increase as the PCR amplification proceeds. The ROX fluorescence is included in the PCR Master Mix as a reference dye to normalize the FAM signal.
2-8. The number of PCR cycle that reached to the threshold fluorescence intensity was defined as threshold cycle (C_{T}). The C_{T} value was determined by using StepOne software version 2.3 (Thermo Scientific).
2-9. Standard curve was generated by plotting C_{T} values in the Y-axis, and the quantity of template DNA (copies/μL) in the X-axis (FIG. 4). The linear regression line was generated as the standard curve for qPCR. The linear dynamic range of the standard curve was broad, ranging from 2 to 2×10⁷ copies/μL, with high goodness-of-fit (r²) value (r²=0.997).
2-10. The PCR amplification efficiency (E_{Amp}) was calculated from the slope (S) of the regression line and the following equation: E_{Amp}=10^{(-1/S)}−1. From this equation, the E_{Amp} of this assay was 98.4%, which is within optimal range (90-110%) (Bustin et al., 2009).

TABLE 1
The qPCR assays developed in this study.

| Assay | Primer and probe name | Sequence (5′->3′) |
|---|---|---|
| 1 | NH8B_3960tnp_1841F | CTGGCTGTCTAGGCCCTGTCT |
| | | (SEQ ID NO: 1) |
| | NH8B_3960tnp_1901R | CCTGCAGGCATGCAAGCT |
| | | (SEQ ID NO: 2) |
| | NH8B_3960tnp_1863MGB | FAM-TTATACACATCTCAACCC |
| | | TG-NFQ-MGB |
| 2 | NH8B_3960tnp_3629F | CATCGATGATGGTTGAGATGTGT |
| | | (SEQ ID NO: 3) |
| | NH8B_3960tnp_3707R | CCCAAATGATCAGTTAAGTGGTA |
| | | AAC (SEQ ID NO: 4) |
| | NH8B_3960tnp_3659MGB | FAM-ACAGGTCTAGGCCTTC- |
| | | NFQ-MGB |

TABLE 2
Composition of the qPCR master mix.

| | Volume to add (μL) | |
|---|---|---|
| | per reaction | Final conc. |
| 2× FastStart Universal Probe Master | 5 | 1× |
| Forward primer (50 μM) | 0.2 | 1 μM |
| Reverse primer (50 μM) | 0.2 | 1 μM |
| TaqMan probe (10 μM) | 0.08 | 80 nM |
| Nuclease-free water | 3.52 | — |
| DNA template | 1 | — |

### Example 3. Specific Quantification of NH8B-1D2

This example shows the specific quantification of NH8B-1D2 by using conventional qPCR.
3-1. Strain NH8B (wild type strain; Tago et al 2011) was grown in R2A broth at 30° C. Strain NH8B-1D2 (mutant strain, see Example 1) was grown in R2A broth (Teknova) supplemented with 100 μg/L kanamycin at 30° C. In addition, 36 non-target bacteria strains listed in Table 3 were grown in the media as described previously (Ishii et al 2013).
3-2. After 24-h growth, cell suspension was pelleted by centrifugation at 10,000 ×g for 5 min.
3-3. DNA was extracted using DNeasy Blood & Tissue kit (Qiagen) according to the manufacturer's instruction.
3-4. The qPCR master mix was prepared as described above (2-5).
3-5. DNA extracted in Step 2-4 was used as the template for qPCR reaction. In addition to the sample DNA, plasmid DNA standards prepared as described above (2-2, 2-3, and 2-4) were subject for qPCR.
3-6. The qPCR and the data analysis were done as described above (2-6 and 2-7).
3-7. The FAM fluorescence signal was obtained only from NH8B-1D2 strain. Other bacterial strains, including NH8B wild type strain, did not show amplification with these assays (NH8B_3960tnp1 and NH8B_3960tnp2), suggesting the assays are very specific to NH8B-1D2.

TABLE 3
Bacteria strains tested in this study.

| Species (serotype/serovar) | Strain ID |
|---|---|
| Enterococcus faecalis | JCM 5803^{T} |
| Enterococcus faecalis | JCM 7783 |
| E. coli K12 | MG1655 |
| E. coli O157:H7 | RIMD 0509952 (Sakai) |
| E. coli O157:H7 | LMG 21756 |
| E. coli O111:HUT | RIMD 05092017 |
| E. colli O26:H11 | RIMD 05091992 |
| Enteroinvasive E. coli | RIMD 0509763 |
| Shigella flexneri 5a | RIMD 3102037 |
| Shigella flexneri 1a | RIMD 3102002 |
| Shigella sonnei | RIMD 104005 |
| Salmonella Typhimurium | JCM 1652^{T} |
| Salmonella Typhimurium | JCM 6977 |
| Salmonella Typhimurium | JCM 6978 |
| Campylobacter jejuni | JCM 2013 |
| Campylobacter coli | JCM 2529^{T} |
| Campylobacter lari | JCM 14870^{T} |
| Clostridium perfringens | JCM 1290^{T} |
| Clostridium perfringens | F4649 |
| Clostridium perfringens | NTCT 8239 |
| Legionella pneumophila | JCM 7571^{T} |
| Listeria monocytogenes serovar 1/2a | JCM 7671 |
| Listeria monocytogenes serovar 1/2c | JCM 7672 |
| Listeria monocytogenes serovar 3a | JCM 7673 |
| Listeria monocytogenes serovar 4a | JCM 7674 |
| Listeria monocytogenes serovar 4b | JCM 7675 |
| Listeria monocytogenes serovar 1/2b | JCM 7676 |
| Listeria monocytogenes serovar 3b | JCM 7677 |
| Listeria monocytogenes serovar 3c | JCM 7678 |
| Listeria monocytogenes serovar 4c | JCM 7679 |
| Listeria monocytogenes serovar 4d | JCM 7680 |
| Vibrio cholerae O1 | RIMD 2203246 |
| Vibrio cholerae O1 | RIMD 2203938 |
| Vibrio cholerae O139 | RIMD 2214451 |
| Vibrio parahaemolyticus | RIMD 2210633 |
| Vibrio paraheamolyticus EB101 | RIMD 2210001 |
| Pseudogulbenkiania sp. | NH8B |
| Pseudogulbenkiania sp. | NH8B-1D2 |

Example 4. Application of the NH8B-1D2 Assay for Microfluidic qPCR
This example shows the NH8B-1D2 assay as described in Example 1 can be applied to the microfluidic qPCR (MFQPCR) system (Fluidigm BioMark HD system). Multiple qPCR assays simultaneously on a chip in the MFQPCR system (Ishii et al 2013). Prior to the MFQPCR, a specific target amplification (STA) reaction was performed to increase the amount of DNA template. The STA reaction is a 14-cycle multiplex PCR with all primers used for the MFQPCR (Spurgeon et al 2008). Both DNA samples and the standard plasmid mixture were subjected to the STA reaction.
4-1. Twenty four assays, including NH8B_3960tnp1, were selected for this example. The primer and probe information is shown in Table 4. Forward and reverse primers of each assay were mixed at a final concentration of 20 μM to prepare a Primer Pair Mix solution. The 1μL each of the 24 Primer Pair Mix solutions was mixed in a single tube containing 76 μL nuclease free water (i.e., a final volume of 100 μL). The final concentration of each primer becomes 0.2 μM. This solution was named STA Primer Pool.
4-2. For each assay, linearized plasmid DNA solution was prepared as described previously (Ishii et al 2013).
4-3. All 24 plasmids were mixed together at final concentration of 2×10⁶ copies/μL, each (Std_2E6 solution). The Std_2E6 solution was serially diluted to 2×10⁵, 2×10⁴, 2×10³, 200, 20, and 2 copies/μL, (Std_2E5, Std_2E4, Std_2E3, Std_2E2, Std_2E1, Std_2E0 respectively).
4-4. The STA reaction mixture (100 μL) contained 5μL of 2× TaqMan PreAmp master mix (Applied Biosystems), 2.5 μL of STA Primer Pool, and 2.5 μL of the DNA template. The reaction was performed with StepOnePlus Real-Time PCR system (Thermo Scientific) with the following conditions: 95° C. for 10 min, followed by 14 cycles at 95° C. for 15 sec, and 60° C. for 4 min.
4-5. After STA reaction, the PCR products (10 μL) were diluted five fold by adding 40 of TE buffer (10 mM Tris-HCl and 0.1 mM EDTA [pH=8]) and used for the MFQPCR.
4-6. The MFQPCR was performed in duplicate using BioMark HD reader (Fluidigm, South San Francisco, Calif.) with a Dynamic Array 48.48 chip (Fluidigm). The sample pre-mix (5 μL) for the MFQPCR reaction contained 2.5 μL of 2× TaqMan Universal PCR Master Mix (Thermo Scientific), 0.25 μL of the 20× GE Sample Loading Reagent (Fluidigm), and 2.25 μL five-fold diluted STA product. The assay pre-mix (5 μL) contained 2.5 μL of 2× Assay Loading Rreagent (Fluidigm), 2 μL of Primer Pair Mix, and 0.5 μL of 10 μM probe. The sample pre-mix and assay-premix were loaded into the Dynamic Array chip, and mixed using an IFC controller according to the manufacturer's instruction. The MFQPCR reaction was run with the following conditions: 50° C. for 2 min, 95° C. for 10 min, and then following 40 cycles with 95° C. for 15 sec, 70° C. for 5 sec, and 60° C. for 60 sec.
4-7. The C_{T} values were determined using Real-Time PCR Analysis software version 4.1.3 (Fludigm). The standard curves were based on linear regression between the C_{q} value and the amount of the template DNA (log copies/μL). The goodness of fit (r²) of each standard curve was analyzed. Based on the slope of each standard curve, the amplification efficiency was also analyzed. The recovery efficiencies were calculated on the basis of the quantity of target gene divided by the quantity of target cells inoculated.
4-8. Standard curve was generated, and r² and E_{Amp}, were calculated for each assay as described above (1-8 and 1-9). Similar to the results obtained by conventional qPCR, broad dynamic range (2 to 2×10⁶ copies/μL), high goodness-of-fit (r²) value (r²=0.998), and good E_{Amp}, value (100.6%) were obtained for NH8B-1D2 assay by MFQPCR (FIG. 5). In addition, the quantitative performances of the other assays were all acceptable (Table 5).

TABLE 4
Primer and probe sequences used in this study.

| | Target | Primer and | | |
|---|---|---|---|---|
| Target organism | gene | probe name | Primer and probe sequence (5′->3′) | Reference |
| Enterococcus spp. | 23S | ECST748F | GAGAAATTCCAAACGAACTTG (SEQ ID NO: 5) | Ludwig |
| | rRNA | ENC854R | CAGTGCTCTACCTCCATCATT (SEQ ID NO: 6) | and |
| | | GPL813TQ | FAM-TGGTTCTCT/ZEN/CCGAAATAGCTTTAGGGCTA- | Schleifer |
| | | | IBFQ | 2000 |
| General E. coli | ftsZ | ftsZ_973F | CTGGTGACCAATAAGCAGGTT (SEQ ID NO: 7) | Ishii et al |
| | | ftsZ_1032R | CATCCCATGCTGCTGGTAG (SEQ ID NO: 8) | 2013 |
| | | UPL71 | | |
| | uidA | uidA_993F | CCCTTACGCTGAAGAGATGC (SEQ ID NO: 9) | Ishii et al |
| | | uidA_1053R | TTCATCAATCACCACGATGC (SEQ ID NO: 10) | 2013 |
| | | UPL113 | | |
| Enteropathogenic | eaeA | eaeA_877F | GGCGAATACTGGCGAGACTA (SEQ ID NO: 11) | Ishii et al |
| E. coli EPEC | | eaeA_976R | GGCGCTCATCATAGTCTTTCTT (SEQ ID NO: 12) | 2013 |
| | | UPL28 | | |
| Shiga-toxin | stx₁ | stx1_636F | GCGTGGGTATTAATGAGTTGG (SEQ ID NO: 13) | Ishii et al |
| producing E. coli | | stx1_711R | TCATCTCGTTCAGTACGGTGTATT (SEQ ID NO: 14) | 2013 |
| STEC | | UPL60 | | |
| | stx₂ | stx2_483F | TGTAATGACTGCTGAAGATGTTGAT (SEQ ID NO: 15) | Ishii et al |
| | | stx2_560R | TCCATGATARTCAGGCAGGA (SEQ ID NO: 16) | 2013 |
| | | UPL126 | | |
| Shigella spp. | ipaH 7.8 | ipaH_81FF | TCTGAGAATCCTGACTGAATGG (SEQ ID NO: 17) | Ishii et al |
| | | ipaH_142R | AAGCAATGCCTCGCTCTTC (SEQ ID NO: 18) | 2013 |
| | | UPL7 | | |
| | ipaH all | ipaH_1136F | AAGGCCTTTTCGATAATGATACC (SEQ ID NO: 19) | Ishii et al |
| | | ipaH_1202R | ATTTCGAGGCGGAACATTT (SEQ ID NO: 20) | 2013 |
| | | UPL108 | | |
| Shigella flexneri | virA | virA_836F | GGCAATCTCTTCACATCACG (SEQ ID NO: 21) | Ishii et al |
| | | virA_897R | TTCGGACATAATTTGGGCATA (SEQ ID NO: 22) | 2013 |
| | | UPL6 | | |
| Campylobacter | cadF | cadF_267F | TGCTATTAAAGGTATTGATGTRGGTGA (SEQ ID NO: 23) | Ishii et al |
| jejuni | | cadF_350R | GCAGCATTTGAAAAATCYTCAT (SEQ ID NO: 24) | 2013 |
| | | UPL39 | | |
| | ciaB | ciaB_718F | GCGTTTTGTGAAAAAGATGAAGATAG (SEQ ID NO: 25) | Ishii et al |
| | | ciaB_797R | GGTGATTTTACTTTCATCCAAGC (SEQ ID NO: 26) | 2013 |
| | | UPL137 | | |
| Campylobacter lari | bipA | Campy2fCla | CATTTCAGCTTTTCTTTTGCCTAGT (SEQ ID NO: 27) | Bonjoch et |
| | | Campy2rCla | AAAACCGAACCATTTGAACACTTAG (SEQ ID NO: 28) | al 2010 |
| | | CAMPY2pr | FAM-ACCACACCA/ZEN/GTAAAATCATCAGGCACATCA- | |
| | | | IBFQ | |
| Salmonella | invA | invA_176F | CAACGTTTCCTGCGGTACTGT (SEQ ID NO: 29) | Gonzalez- |
| Typhimurium | | invA_291R | CCCGAACGTGGCGATAATT (SEQ ID NO: 30) | Escalona |
| | | invA_FAM 208 | FAM-CTCTTTCGT/ZEN/CTGGCATTAT-IBFQ | et al 2009 |
| | ttrC | ttrC_440F | ATTTTTGGCAGCCTTACCG (SEQ ID NO: 31) | Ishii et al |
| | | ttrC_507R | GCCTTACAGGCGTTCTTCG (SEQ ID NO: 32) | 2013 |
| | | UPL149 | | |
| Clostridium | 16S | CPerf165F | CGCATAACGTTGAAAGATGG (SEQ ID NO: 33) | Wise and |
| perfringens | rRNA | CPerf269R | CCTTGGTAGGCCGTTACC (SEQ ID NO: 34) | Siragusa |
| | | CPerf187FAM | FAM-TCATCATTC/ZEN/AACCAAAGGAGCAATCC-IBFQ | 2005 |
| | cpe | cpe_823F | GAACAGTCCTTAGGTGATGGAGTAA (SEQ ID NO: 35) | Ishii et al |
| | | cpe_914R | GATGAATTAGCTTTCATTACAAGAACA (SEQ ID NO: 36) | 2013 |
| | | UPL159 | | |
| Legionella | mip | mip_99F | GGATAAGTTGTCTTATAGCATTGGTG (SEQ ID NO: 37) | Ishii et al |
| pneumophila | | mip_172R | CCGGATTAACATCTATGCCTTG (SEQ ID NO: 38) | 2013 |
| | | UPL60 | | |
| Listeria | iap | iap_1359F | TGGCGTTAAATACGATAACATCC (SEQ ID NO: 39) | Ishii et al |
| monocytogenes | | iap_1421R | CGACCGAAGCCAACTAGATATT (SEQ ID NO: 40) | 2013 |
| | | UPL106 | | |
| | hlyA | Lm_hlyA_232F | TACCACGGAGATGCAGTGAC (SEQ ID NO: 41) | Ishii et al |
| | | Lm_hlyA308R | TTCTCCACAACAATATATTCATTTCC (SEQ ID NO: 42) | 2013 |
| | | UPL142 | | |
| Vibrio cholera | ctxA | VC_ctxAF | TTTGTTAGGCACGATGATGGAT (SEQ ID NO: 43) | Blackstone |
| | | VC_ctxAR | ACCAGACAATATAGTTTGACCCACTAAG (SEQ ID NO: 44) | et al 2007 |
| | | VC_ctxA_MGB | FAM-TGTTTCCAC/ZEN/CTCAATTAGTTTGAGAAGTGCCC- | |
| | | | IBFQ | |
| | toxR | VC_toxR_420/ | GTTTGGCGWGAGCAAGGTTT (SEQ ID NO: 45) | Liu et al |
| | | 334F | | 2012 |
| | | VC_toxR_585R | TCTCTTCTTCAACCGTTTCCA (SEQ ID NO: 46) | |
| | | toxR_464/ | FAM-CGCAGAGTM/ZEN/GAAATGGCTTGG-IBFQ | |
| | | 378FAM | | |
| Vibrio | tdhS | VP_tdhF | AAACATCTGCTTTTGAGCTTCCA (SEQ ID NO: 47) | Blackstone |
| paraheamolyticus | | VP_tdhR | CTCGAACAACAAACAATATCTCATCAG (SEQ ID NO: 48) | et al 2003 |
| | | VP_tdhS_MGB | FAM-TGTCCCTTT/ZEN/TCCTGCCCCCGG-IBFQ | |
| Pseudogulbenkiania | NH8B_3641 | IAC_23F | CAGGCCGTGAAGTCAAGC (SEQ ID NO: 49) | Ishii et al |
| sp. NH8B | | IAC_92R | GAGGCGATGTGGATGGTC (SEQ ID NO: 50) | 2013 |
| | | UPL56 | | |
| Pseudogulbenkiania | NH8B_ | NH8B_3960tnp_ | CTGGCTGTCTAGGCCCTGTCT (SEQ ID NO: 51) | This study |
| sp. NH8B-1D2 | 3960tnp | 1841F | | |
| | | NH8B_3960tnp_ | CCTGCAGGCATGCAAGCT (SEQ ID NO: 52) | |
| | | 1901R | | |
| | | NH8B_3960tnp_ | FAM-TTATACACATCTCAACCCTG-NFQ-MGB | |
| | | 1863 | | |

Blackstone etal., 2003, Journal of Microbiological Methods 53: 149-155; Blackstone etal., 2007, Journal of Microbiological Methods 68: 254-259; Bonjoch et al., 2010, Food Anal Methods 3: 40-46; Gonzalez-Escalona et al., 2009, Applied and Environmental Microbiology 75: 3714-3720; Ishii et al., 2013, Applied and Environmental Microbiology 79: 2891-2898; Liu et al., 2012, Journal of Clinical Microbiology 50: 98-103; Ludwig and Schleifer, 2000, Systematic and Applied Microbiology 23: 556-562; Wise and Siragusa, 2005, Applied and Environmental Microbiology 71: 3911-3916.

TABLE 5
Quantitative performance of the conventional
qPCR and MFQPCR assays.

| | Target | Meth- | Inter- | | | |
|---|---|---|---|---|---|---|
| Target organism | gene | od^{a} | cept | Slope | r² | E_{AMP} |
| Enterococcus spp. | 23S | A | 40.0 | −3.62 | 0.998 | 89.0 |
| | rRNA | B | 23.4 | −3.22 | 0.998 | 104.4 |
| General E. coli | ftsZ | A | 38.8 | −3.37 | 0.992 | 98.2 |
| | | B | 26.5 | −3.09 | 0.996 | 110.6 |
| | uidA | A | 40.4 | −3.77 | 0.997 | 84.1 |
| | | B | 26.0 | −3.38 | 0.997 | 97.6 |
| Enteropathogenic E. coli | eaeA | A | 38.6 | −3.61 | 0.998 | 89.3 |
| EPEC | | B | 25.9 | −3.36 | 0.997 | 98.6 |
| Shiga-toxin producing | stx₁ | A | 37.8 | −3.34 | 0.964 | 99.1 |
| E. coli STEC | | B | 26.1 | −3.35 | 0.995 | 98.6 |
| | stx₂ | A | 40.9 | −3.54 | 0.997 | 91.6 |
| | | B | 27.0 | −2.99 | 0.996 | 116.1 |
| Shigella spp. | ipaH | A | 37.2 | −3.80 | 0.997 | 83.3 |
| | 7.8 | B | 23.6 | −3.27 | 0.999 | 102.0 |
| | ipaH | A | 38.3 | −3.78 | 0.996 | 83.9 |
| | all | B | 23.5 | −3.25 | 0.996 | 103.1 |
| Shigella flexneri | virA | A | 39.8 | −3.57 | 0.999 | 90.6 |
| | | B | 26.3 | −3.10 | 0.995 | 101.7 |
| Campylobacter jejuni | cadF | A | 42.3 | −3.46 | 0.982 | 94.7 |
| | | B | 25.8 | −3.34 | 0.975 | 96.0 |
| | ciaB | A | 40.2 | −3.63 | 0.998 | 88.5 |
| | | B | 28.8 | −3.33 | 0.993 | 99.8 |
| Campylobacter lari | bipA | A | 40.0 | −3.91 | 0.999 | 80.3 |
| | | B | 25.4 | −3.47 | 0.994 | 94.0 |
| Salmonella Typhimurium | invA | A | 42.9 | −3.91 | 0.997 | 80.2 |
| | | B | 27.6 | −3.52 | 0.989 | 92.2 |
| | ttrC | A | 38.4 | −3.69 | 0.997 | 86.5 |
| | | B | 29.3 | −3.39 | 0.996 | 97.1 |
| Clostridium perfringens | 16S | A | 41.4 | −3.89 | 1.000 | 80.6 |
| | rRNA | B | 26.4 | −3.16 | 0.992 | 107.4 |
| | cpe | A | 41.2 | −3.66 | 0.997 | 87.6 |
| | | B | 27.0 | −3.55 | 0.989 | 91.4 |
| Legionella pneumophila | mip | A | 37.9 | −3.52 | 0.998 | 92.3 |
| | | B | 25.0 | −3.28 | 0.995 | 101.7 |
| Listeria monocytogenes | iap | A | 39.4 | −3.71 | 0.995 | 85.9 |
| | | B | 23.9 | −3.20 | 0.998 | 105.3 |
| | hlyA | A | 46.9 | −3.48 | 0.988 | 94.0 |
| | | B | 29.5 | −3.01 | 0.979 | 114.8 |
| Vibrio cholerae | ctxA | A | 38.7 | −3.93 | 0.998 | 79.7 |
| | | B | 24.4 | −3.39 | 0.997 | 97.1 |
| | toxR | A | 42.4 | −3.86 | 0.998 | 81.7 |
| | | B | 27.1 | −3.42 | 0.996 | 96.2 |
| Vibrio paraheamolyticus | tdh | A | 36.5 | −3.72 | 0.995 | 85.6 |
| | | B | 22.7 | −3.15 | 0.997 | 107.8 |
| Pseudogulbenkiania sp. | NH8B_{—} | A | 37.2 | −3.35 | 0.994 | 98.7 |
| NH8B | 3641 | B | 25.0 | 3.33 | 0.997 | 99.7 |
| Pseudogulbenkiania sp. | NH8B_{—} | A | 40.8 | −3.36 | 0.997 | 98.4 |
| NH8B-1D2 | 3960tnp | B | 29.5 | −3.31 | 0.998 | 100.6 |

^{a}Method: A, conventional qPCR; B, microfluidic qPCR MFQPCR

### Example 5. Calculation of the DNA Recovery Efficiencies

This example shows how to calculate DNA recovery efficiencies of the sample process control strains (e.g., Pseudogulbenkiania sp. strain NH8B-1D2).
5-1. Strain NH8B-1D2 strain was grown in R2A broth supplemented with 100 82 g/L kanamycin at 30° C. for overnight.
5-2. Optical density at 600 nm (0D₆₀₀) of NH8B-1D2 cells was measured using a spectrophotometer. The OD₆₀₀ value was converted to cell concentrations (C_{SPC} ) using the equation (Eq. 1). This equation can be optimized by counting the number of cells directly under microscope.

C_{SPC}=OD₆₀₀×0.5×10⁸ Eq. 1
5-3. The NH8B-1D2 cells were serially diluted to 5×10⁷, 5×10⁶, 5×10⁵, 5×10⁴, 5×10³, 500, and 50 cells/mL with PBS buffer. These cell suspensions are named NH8B-1D2 inocula.
5-4. NH8B-1D2 inoculum (1 mL each) was spiked to 5 L each of pond water. The final concentration of NH8B-1D2 becomes 10⁷, 10⁶, 10⁵, 10⁴, 10³, 100, and 10 cells per liter of pond water, respectively. These concentrations were named as I_{SPC} (i.e., the concentration of the sample process control strain spiked to the environmental samples). As a negative control, 5L of pond water without inoculation of bacterium was also prepared.
5-5. Spiked pond water (5 L) was filtered through 0.22-μm-pore polyethersulfone membrane filter (Millipore) as previously described in detail (Ishii et al 2014).
5-6. The membrane was cut intro eight pieces and placed in a 50-mL plastic centrifuge tube.
5-7. The membrane pieces were vigorously shaken in 20 mL phosphate buffered saline (pH 7.2) containing 0.1% gelatin (Hamilton et al 2010). This process allows us to detach microbial cells from the membrane.
5-8. The cell suspension was pelleted by centrifugation at 10,000 × g for 15 min. The pellet was re-suspended with 1 mL PBS.
5-9. All cell suspension was transferred to a new 1.5 mL microcentrifuge tube, and pelleted by centrifugation at 10,000 × g for 5 min.
5-10. DNA was extracted from the cell pellet by using PowerSoil DNA Isolation Kit (MoBio) according to the manufacturer's instruction.
5-11. The DNA samples were used for the MFQPCR as described in Example 4. By using the standard curve the DNA concentration (copies/μL) of the samples can be calculated based on their C_{T} value. Because the target gene is present per genome (=per cell) by design, copies/μL can be converted to cells/μL.
5-12. The DNA recovery efficiency of the sample process control strain (E_{SPC}) was calculated using the equation (Eq. 2).

E_{SPC}=Q_{SPC}/I_{SPC}×100 Eq. 2

where Q_{SPC} is the concentration of the sample process control (=NH8B-1D2) measured by qPCR and I_{SPC} is the concentration of the sample process control strain spiked to the environmental samples (i.e., in this example, a pond water). The unit of E_{SPC} is percentage (%).
5-13. The average (±standard deviation) of E_{SPC} was 12.4±5.8% based on the MFQPCR. This indicates that about 80-90% of DNA was lost during DNA extraction process.

### Example 6. Use of Strain NH8B-1D2 as the Sample Process Control

This example shows how Strain NH8B-1D2 can be used as the sample process control (SPC) strain to normalize DNA recovery efficiencies of pathogens. The strains used were Escherichia coil O157:H7 strain Sakai (RIMD 0509952), Salmonella enterica serovar Typhimurium JCM 1652^{T}, Campylobacter jejuni JCM 2013^{T}, and Listeria monocytogenes serovar 1/2a JCM 7671. While E. coli, Salmonella Typhymurium, and Campylobacter jejuni are Gram-negative bacteria, Listeria monocytogenes is Gram-positive bacterium.
6-1. Pathogen spike experiments were conducted in a similar manner as described in Example 5. In brief, pathogen (one of the four pathogens listed above) and Strain NH8B-1D2 were co-spiked to the pond water, at concentration of each bacterium ranging from 10 to 10⁷ cells per liter of pond water. The spiked pond water was filtered, and cell pellets were prepared as described above (5-5-5.9). DNA was extracted and MFQPCR was performed as described above (5-10 and Example 4). By using MFQPCR, it is possible to detect and quantify multiple target bacteria, including the SPC strain, simultaneously (FIG. 6).
6-2. The DNA recovery efficiency of each pathogen and SPC (E_{PATH} and E_{SPC}, respectively) was calculated in a similar manner as described in 5-12. Average DNA recovery efficiencies obtained in this experiment was named as E′_{PATH} and E_{SPC} for each pathogen and SPC, respectively (Table 6). From this table, ratio in the DNA recovery efficiencies (RE_{PATH/SPC}) were calculated using the equation (Eq. 3) for each pathogen.

RE_{PATH/SPC}=E′_{PATH}/E′_{SPC} Eq. 3
6-3. The RE_{PATH/SPC} were calculated using E′_{PATH} and E_{SPC} values, which are the average values of E_{PATH} and E_{SPC} obtained from multiple experiments with different concentrations of pathogens. Because large variation in E_{PATH} and E_{SPC} was not observed, it is valid to treat RE_{PATH/SPC} values as specific constant for each pathogen. Based on this assumption, the E_{PATH} of environmental samples with unknown real concentrations of target pathogens can be calculated by using the equation (Eq. 4).

E_{PATH}=RE_{PATH/SPC}×E_{SPC}
6-4. The E_{PATH} values were calculated for each pathogen (Escherichia coli O157:H7 strain Sakai (RIMD 0509952), Salmonella enterica serovar Typhimurium JCM1652^{T}, Campylobacter jejuni JCM2013^{T}, and Listeria monocytogenes serovar 1/2a JCM 7671).
6-5. By using E_{PATH} values, the quantity of pathogens in the original samples (Q^{N}PATH) can be normalized by using the equation (Eq. 4).

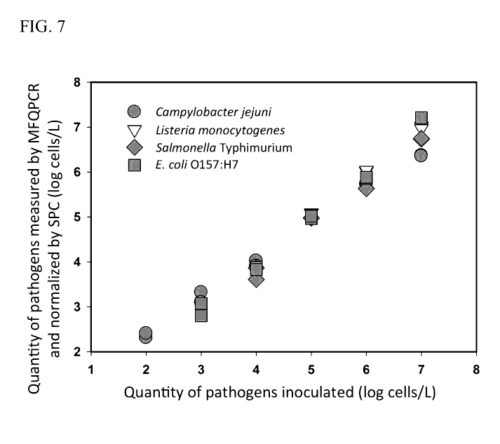

Q^{N}_{PATH}=Q_{PATH}/E_{PATH}×100 Eq. 4

where Q_{PATH} is the quantity of pathogens measured by MFQPCR.
6-6. To test if Q^{N}_{PATH} values calculated by Eq. 4 were similar to the actual quantity of pathogens, Q^{N}PATH and I_{PATH} (i.e., the concentration of pathogens spiked to the environmental samples) values were compared. Results shown in FIG. 7 and Table 7 suggest that Q^{N}_{PATH} and I_{PATH} values were very similar. Therefore, we conclude that it is technically feasible to normalize pathogen concentrations by using SPC such as Pseudogulbenkiania sp. NH8B-1D2 strain.

TABLE 6
Average DNA recovery efficiencies of pathogens and
SPC E′_{PATH} and E′_{SPC}, respectively.

| | DNA recovery | Ratio in the |
|---|---|---|
| | efficiency | DNA recovery |
| Bacteria | (mean ± SD) | efficiencies (RE_{PATH/SPC}) |
| E. coli O157:H7 | 10.1 ± 4.0 | 0.8 |
| Salmonella Typhimurium | 9.2 ± 3.6 | 0.7 |
| Campylobacter jejuni | 70.9 ± 17.6 | 5.7 |
| Listeria monocytogenes | 7.8 ± 0.3 | 0.6 |
| Pseudogulbenkiania sp. | | |
| NH8B-1D2 | 12.4 ± 5.8 | |

TABLE 7
Slopes and intercepts of the linear regression equations between concen-
trations of pathogens measured by MFQPCR and normalized by using SPC
Q^{N}_{PATH} and the actual concentrations of pathogens I_{PATH}; the concen-
tration of pathogens spiked to the environmental samples. Goodness-
of-fit (r²) values are also shown.

| Pathogen | Slope | Intercept | r² |
|---|---|---|---|
| E. coli O157:H7 | 1.03 | −0.1 | 0.999 |
| Salmonella Typhimurium | 0.98 | 0.1 | 0.989 |
| Campylobacter jejuni | 1.02 | −0.2 | 0.999 |
| Listeria monocytogenes | 0.99 | 0.4 | 1.000 |

^{a} Slope (a) and intercept (b) of a linear regression equation (y = ax + b) is shown for each pathogen.

### Citations for Examples 1-6

Bustin S A, Benes V, Garson J A, Hellemans J, Huggett J, Kubista M et al (2009). The MIQE Guidelines: Minimum Information for Publication of Quantitative Real-Time PCR Experiments. Clinical Chemistry 55: 611-622.

Hamilton M J, Hadi A Z, Griffith J F, Ishii S, Sadowsky M J (2010). Large scale analysis of virulence genes in Escherichia coli strains isolated from Avalon Bay, Calif. Water Research 44: 5463-5473.

Ishii S, Tago K, Nishizawa T, Oshima K, Hattori M, Senoo K (2011). Complete Genome Sequence of the Denitrifying and N2O-Reducing Bacterium Pseudogulbenkiania sp. Strain NH8B. Journal of Bacteriology 193: 6395-6396.

Ishii S, Segawa T, Okabe S (2013). Simultaneous Quantification of Multiple Food- and Waterborne Pathogens by Use of Microfluidic Quantitative PCR. Applied and Environmental Microbiology 79: 2891-2898.

Ishii S, Nakamura T, Ozawa S, Kobayashi A, Sano D, Okabe S (2014). Water Quality Monitoring and Risk Assessment by Simultaneous Multipathogen Quantification. Environmental Science & Technology 48: 4744-4749.

Larsen R, Wilson M, Guss A, Metcalf W (2002). Genetic analysis of pigment biosynthesis in Xanthobacter autotrophicus Py2 using a new, highly efficient transposon mutagenesis system that is functional in a wide variety of bacteria. Archives of Microbiology 178: 193-201.

Saltikov C W, Newman D K (2003). Genetic identification of a respiratory arsenate reductase. Proceedings of the National Academy of Sciences 100: 10983-10988.

Spurgeon S L, Jones R C, Ramakrishnan R (2008). High Throughput Gene Expression Measurement with Real Time PCR in a Microfluidic Dynamic Array. PLoS ONE 3: e1662.

Tago K, Ishii S, Nishizawa T, Otsuka S, Senoo K (2011). Phylogenetic and Functional Diversity of Denitrifying Bacteria Isolated from Various Rice Paddy and Rice-Soybean Rotation Fields. Microbes and Environments 26: 30-35.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for determining the concentration of a microbe in a sample comprising:
extracting DNA of microbes present in a sample, wherein the sample comprises a known number of cells of a genetically engineered microbe, and wherein the sample is suspected of comprising a test microbe;
adding primers to the sample, wherein the primers comprise (1) primers to amplify DNA of the genetically engineered microbe and (2) primers to amplify DNA of the test microbe;
exposing the sample to conditions suitable for amplification of a target polynucleotide, wherein the target polynucleotide comprises a target polynucleotide of the genetically engineered microbe, a target polynucleotide of the test microbe, or a combination thereof;
determining the DNA recovery efficiency of the genetically engineered microbe; and
calculating the number of cells of the test microbe in the sample.

2. The method of claim 1 further comprising adding to the sample a known number of cells of the genetically engineered microbe.

3. The method of claim 1 further comprising concentrating the microbes present in the sample.

4. The method of claim 1 wherein the genetically engineered microbe and the test microbe are Gram negative microbes.

5. The method of claim 1 wherein the genetically engineered microbe and the test microbe are Gram positive microbes.

6. The method of claim 1 wherein the sample is divided into at least two aliquots, and the primers that amplify DNA of the genetically engineered microbe are added to one aliquot and the primers that amplify DNA of the test microbe are added to a second aliquot. The method of claim 1 wherein the sample comprises an environmental sample.

8. The method of claim 7 wherein the environmental sample comprises recreational water.

9. The method of claim 8 wherein the recreational water comprises ocean water, pond water, lake water, creek water, river water, swimming pool water, hot tub water, or sauna water.

10. The method of claim 1 wherein the sample comprises a clinical sample.

11. The method of claim 10 wherein the clinical sample comprises tissue, stool, or a body fluid.

12. The method of claim 11 wherein the body fluid comprises cerebrospinal fluid, blood, urine, sputum, or synovial fluid.

13. The method of claim 12 wherein the sample comprises a food sample.

14. The method of claim 13 wherein the food sample comprises meat, fish, mild, cheese, fruit or vegetable.

15. The method of claim 1 wherein the sample comprises groundwater, leachate, wastewater, sewer water, blackwater, graywater, bilge water, ballast water, feed water, process water, industrial water, irrigation water, rain water, runoff water, cooling water, nonpotable water, potable water, or drinking water.

16. The method of claim 1 wherein the sample does not comprise a test microbe.

17. A method for determining the concentration of a microbe in a sample comprising:
extracting DNA of microbes present in a sample, wherein the sample comprises a known number of cells of a genetically engineered microbe, and wherein the sample is suspected of including a test microbe;
adding primers to the sample, wherein the primers comprise (1) primers to amplify DNA of the genetically engineered microbe and (2) primers to amplify DNA of the test microbe;
amplifying a target polynucleotide of the genetically engineered microbe and a target polynucleotide of the test microbe;
determining the DNA recovery efficiency of the genetically engineered microbe;
calculating the number of cells of the test microbe in the sample.

18. A kit comprising in separate containers:
a genetically engineered microbe, wherein the microbe is not a member of the microbiota of a human or an animal;
primers to amplify a target polynucleotide present in the genetically engineered microbe; and
primers to amplify a test microbe.

19. The kit of claim 18 wherein the test microbe is Campylobacter jejuni, Campylobacter lari, Listeria monocytogenes, Salmonella spp., Shigella spp., Clostridium perfringens, Legionella pneumophila, Listeria monocytogenes, Vibrio cholera, Vibrio paraheamolyticus, or E. coli.

20. The kit of claim 19 wherein the E. coli is E. coli O157:H7, a Shiga-toxin producing E. coli, or an enteropathogenic E. coli.

21. The kit of claim 18 wherein the test microbe is a microbe indicative of fecal contamination of a water sample.

22. The kit of claim 21 wherein the test microbe is Enterococcus spp.


SEQUENCE LISTING (53 sequences)

SEQ ID NO: 1 - DNA, 21 bp, artificial
  primer
ctggctgtctaggccctgtct

SEQ ID NO: 2 - DNA, 18 bp, artificial
  primer
cctgcaggcatgcaagct

SEQ ID NO: 3 - DNA, 23 bp, artificial
  primer
catcgatgatggttgagatgtgt

SEQ ID NO: 4 - DNA, 26 bp, artificial
  primer
cccaaatgatcagttaagtggtaaac

SEQ ID NO: 5 - DNA, 21 bp, artificial
  primer
gagaaattccaaacgaacttg

SEQ ID NO: 6 - DNA, 21 bp, artificial
  primer
cagtgctctacctccatcatt

SEQ ID NO: 7 - DNA, 21 bp, artificial
  primer
ctggtgaccaataagcaggtt

SEQ ID NO: 8 - DNA, 19 bp, artificial
  primer
catcccatgctgctggtag

SEQ ID NO: 9 - DNA, 20 bp, artificial
  primer
cccttacgctgaagagatgc

SEQ ID NO: 10 - DNA, 20 bp, artificial
  primer
ttcatcaatcaccacgatgc

SEQ ID NO: 11 - DNA, 20 bp, artificial
  primer
ggcgaatactggcgagacta

SEQ ID NO: 12 - DNA, 22 bp, artificial
  primer
ggcgctcatcatagtctttctt

SEQ ID NO: 13 - DNA, 21 bp, artificial
  primer
gcgtgggtattaatgagttgg

SEQ ID NO: 14 - DNA, 24 bp, artificial
  primer
tcatctcgttcagtacggtgtatt

SEQ ID NO: 15 - DNA, 25 bp, artificial
  primer
tgtaatgactgctgaagatgttgat

SEQ ID NO: 16 - DNA, 20 bp, artificial
  primer
tccatgatartcaggcagga

SEQ ID NO: 17 - DNA, 22 bp, artificial
  primer
tctgagaatcctgactgaatgg

SEQ ID NO: 18 - DNA, 19 bp, artificial
  primer
aagcaatgcctcgctcttc

SEQ ID NO: 19 - DNA, 23 bp, artificial
  primer
aaggccttttcgataatgatacc

SEQ ID NO: 20 - DNA, 19 bp, artificial
  primer
atttcgaggcggaacattt

SEQ ID NO: 21 - DNA, 20 bp, artificial
  primer
ggcaatctcttcacatcacg

SEQ ID NO: 22 - DNA, 21 bp, artificial
  primer
ttcggacataatttgggcata

SEQ ID NO: 23 - DNA, 27 bp, artificial
  primer
tgctattaaaggtattgatgtrggtga

SEQ ID NO: 24 - DNA, 22 bp, artificial
  primer
gcagcatttgaaaaatcytcat

SEQ ID NO: 25 - DNA, 26 bp, artificial
  primer
gcgttttgtgaaaaagatgaagatag

SEQ ID NO: 26 - DNA, 23 bp, artificial
  primer
ggtgattttactttcatccaagc

SEQ ID NO: 27 - DNA, 25 bp, artificial
  primer
catttcagcttttcttttgcctagt

SEQ ID NO: 28 - DNA, 25 bp, artificial
  primer
aaaaccgaaccatttgaacacttag

SEQ ID NO: 29 - DNA, 21 bp, artificial
  primer
caacgtttcctgcggtactgt

SEQ ID NO: 30 - DNA, 19 bp, artificial
  primer
cccgaacgtggcgataatt

SEQ ID NO: 31 - DNA, 19 bp, artificial
  primer
atttttggcagccttaccg

SEQ ID NO: 32 - DNA, 19 bp, artificial
  primer
gccttacaggcgttcttcg

SEQ ID NO: 33 - DNA, 20 bp, artificial
  primer
cgcataacgttgaaagatgg

SEQ ID NO: 34 - DNA, 18 bp, artificial
  primer
ccttggtaggccgttacc

SEQ ID NO: 35 - DNA, 25 bp, artificial
  primer
gaacagtccttaggtgatggagtaa

SEQ ID NO: 36 - DNA, 27 bp, artificial
  primer
gatgaattagctttcattacaagaaca

SEQ ID NO: 37 - DNA, 26 bp, artificial
  primer
ggataagttgtcttatagcattggtg

SEQ ID NO: 38 - DNA, 22 bp, artificial
  primer
ccggattaacatctatgccttg

SEQ ID NO: 39 - DNA, 23 bp, artificial
  primer
tggcgttaaatacgataacatcc

SEQ ID NO: 40 - DNA, 22 bp, artificial
  primer
cgaccgaagccaactagatatt

SEQ ID NO: 41 - DNA, 20 bp, artificial
  primer
taccacggagatgcagtgac

SEQ ID NO: 42 - DNA, 26 bp, artificial
  primer
ttctccacaacaatatattcatttcc

SEQ ID NO: 43 - DNA, 22 bp, artificial
  primer
tttgttaggcacgatgatggat

SEQ ID NO: 44 - DNA, 28 bp, artificial
  primer
accagacaatatagtttgacccactaag

SEQ ID NO: 45 - DNA, 20 bp, artificial
  primer
gtttggcgwgagcaaggttt

SEQ ID NO: 46 - DNA, 21 bp, artificial
  primer
tctcttcttcaaccgtttcca

SEQ ID NO: 47 - DNA, 23 bp, artificial
  primer
aaacatctgcttttgagcttcca

SEQ ID NO: 48 - DNA, 27 bp, artificial
  primer
ctcgaacaacaaacaatatctcatcag

SEQ ID NO: 49 - DNA, 18 bp, artificial
  primer
caggccgtgaagtcaagc

SEQ ID NO: 50 - DNA, 18 bp, artificial
  primer
gaggcgatgtggatggtc

SEQ ID NO: 51 - DNA, 21 bp, artificial
  primer
ctggctgtctaggccctgtct

SEQ ID NO: 52 - DNA, 18 bp, artificial
  primer
cctgcaggcatgcaagct

SEQ ID NO: 53 - DNA, 4705 bp, artificial
  Plasmid pRL27-NH8B-1D2-NcoI-1
aggatcaagcctcacgagcaattagtatcggttagcttcacgcctcacagcgcttccacacccgacctatcaacgtcctggtctcgaacgactcttcaggagggtcaagccctcagggaagtctcatcttcaggcgagtttcccgcttagatgctttcagcggttatctcttccgaacttagctacccggcaatgccactggcgtgacaaccggtacaccagaggttcgtccactccggtcctctcgtactaggagcagcccccgtcaaacttccaacgcccactgcagatagggaccaaactgtctcacgacgttttgaacccagctcacgtaccactttaaatggcgaacagccatacccttgggaccggctacagccccaggatgtgatgagccgacatcgaggtgccaaactccgccgtcgatgtgaactcttgggcggaatcagcctgttatccccggagtaccttttatccgttgagcgatggcccttccattcagaaccaccggatcactatgtcctgctttcgcacctgctcgacttgtcggtctcgcagttaagccaccttatgccattgcactatcagcacgatttccgaccgtacctaggtgaccttcgaactcctccgttacactttgggaggagaccgccccagtcaaactgcctaccatgcactgtccccgatccggatcacggaccaaggttagaacctcaaacacaccagggtggtatttcaaggacggctccactgaaactagcgtctcagcttcatagcctcccacctatcctacacaagtctgttcaaagtccaatgcaaagctacagtaaaggttcacggggtctttccgtctagcagcggggagattgcatcttcacaaacatttcaacttcgctgagtctcaggaggagacagtgtggccatcgttacgccattcgtgcgggtcggaacttacccgacaaggaatttcgctaccttaggaccgttatagttacggccgccgtttaccggggcttcgatcaagagcttgcaccccatcacttaaccttccggcaccgggcaggcgtcacaccgtatacgtccactttcgtgttggcacagtgctgtgtttttgataaacagtcgcagccaccgattctctgcgacctctcgaggcttcagacgcgaggtcctacaccttaagaggcataccttctcccgaagttacggtatcaatttgccgagttccttctcctgagttctctcaagcgccttagaattttcatcctgcccacctgtgtcggtttgcggtacggttctcgtatagctgaagcttagtggcttttcctggaagcgtggtatcagtcacttcggttccgtagaacctcgttatcacgtctcggtgttaacaaaggagcggatttgcctactccttccacctaccggcttgaaccagggcatccaacacctggctgacctaaccttctccgtccccacatcgcactatacgaaagtacgggaatattgacccgtttcccatcgactacgcttttcagcctcgccttaggggccgactcaccctacgccgatgaacgttgcgtaggaaaccttgggctttcggcgagcgggcttttcacccgctttatcgctactcatgtcagcattcgcacttctgatatctccagcatgccttacgacacaccttcacagacctacagaacgctcccctaccatctgcacttgcgtgcaaatccgcggcttcggttatcagtttgagccccgttacatcttccgcgcaggacgactcgaccagtgagctattacgctttctttaaatgatggctgcttctaagccaacatcctggctgtctaggccctgtctcttatacacatctcaaccctgaagcttgcatgcctgcaggtcgactctagatttaaattaattaagagctcgggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccccgagctcatcgatttcgaaccccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggggggtaccgagctcgaattcatcgatgatggttgagatgtgtataagagacaggtctaggccttcccacttcgtttaccacttaactgatcatttgggaccttagccggcggtctgggttgtttccctcttgacgatggacgttagcacccaccgtctgtctcccatgctcgcactttccggtattcagagtttgccatggtttggtaaatcgcaatgaccccctagccataacagtgctttacccccggaagtgatacatgaggcactacctaaatagttttcggggagaaccagctatctccgagtttgtttagcctttcacccctatccacagctcatcccctagttttgcaacactagtgggttcggacctccagtgcgtgttaccgcaccttcatcctggccatggatagatcactcggtttcgggtctacgcccagcaactaaagcgccctattcggactcggtttccctacgcctcccctattcggttaagctcgctactgaacgtaagtcgctgacccattatacaaaaggtacgcagtcaccccttgcgaggctcccactgtttgtatgcatccggtttcaggttctatttcactcccctcccggggttcttttcgcctttccctcacggtactggttcactatcggtcgatcacgagtatttagccttggaggatggtccccccatcttcagacaggatttcgcgtgtcccgccctacttctcgtacgcctagtccttggaatgcgttttcgtgtacggggctatcacccactatggcggccatttccagggccttccactaacacaatccataacacgtacaggctgttccgcgttcgctcgccactactgacggaatctcggttgatttctattcctgcgggtacttagatgtttcagttctccgcgttcgctctaccttgcctatgtgttcagcaaggagtacctattgctaggtgggtttccccattcggatatctccggatcatcgctctattgccagctccccgaagcttttcgcaggcttacacgtccttcatcgcctgtgatcgccaaggcatccaccagatgcacttagtcgcttga